(19) United States
(12) Patent Application Publication
(10) Pub. No.: US 2025/0060731 A1
(43) Pub. Date: Feb. 20, 2025

(54) ENVIRONMENTAL ATTRIBUTES FOR MACHINE FLUIDS AND POLYISOBUTENE

(71) Applicant: BASF SE, Ludwigshafen Am Rhein (DE)

(72) Inventors: Wolfgang Grabarse, Ludwigshafen (DE); Dietmar Haering, Ludwigshafen (DE); Gerhard Schulz, Ludwigshafen (DE); Bastian Grumbrecht, Ludwigshafen (DE); Steffen Thomas Klosterhalfen, Ludwigshafen (DE); Christian Krueger, Ludwigshafen (DE); Kurt Wenzl, Ludwigshafen (DE); Ana Alba Perez, Ludwigshafen (DE); Christopher Alec Anderlohr, Ludwigshafen (DE); Martin Binder, Ludwigshafen (DE); Alessandro Pistillo, Ludwigshafen (DE); Katell Loisel, Ludwigshafen (DE); Sabine Gruettner-Merten, Ludwigshafen (DE); Michael Klos, Ludwigshafen (DE)

(73) Assignee: BASF SE, Ludwigshafen Am Rhein (DE)

(21) Appl. No.: 18/721,512

(22) PCT Filed: Dec. 19, 2022
(86) PCT No.: PCT/EP2022/086638

§ 371 Date: Jun. 18, 2024

Related U.S. Application Data
(60) Provisional application No. 63/416,091, filed on Oct. 14, 2022.

(30) Foreign Application Priority Data
Dec. 21, 2021 (EP) 21216268.9
Dec. 21, 2021 (EP) 21216269.7
Dec. 21, 2021 (EP) 21216270.5
Dec. 21, 2021 (EP) 21216271.3
Dec. 21, 2021 (EP) 21216286.1
Dec. 21, 2021 (EP) 21216292.9
Dec. 21, 2021 (EP) 21216326.5
Dec. 21, 2021 (EP) 21216327.3
Dec. 21, 2021 (EP) 21216333.1
Apr. 4, 2022 (EP) 22166573.0
Apr. 12, 2022 (EP) 22167945.9
May 10, 2022 (EP) 22172609.4
May 10, 2022 (EP) 22172611.0
May 10, 2022 (EP) 22172615.1
May 10, 2022 (EP) 22172617.7
May 10, 2022 (EP) 22172619.3
Sep. 9, 2022 (EP) 22194793.0
Sep. 9, 2022 (EP) 22194800.3
Sep. 9, 2022 (EP) 22194808.6
Sep. 9, 2022 (EP) 22194815.1
Sep. 9, 2022 (EP) 22194818.5
Oct. 14, 2022 (EP) 22201672.7
Oct. 18, 2022 (EP) 22202183.4
Dec. 5, 2022 (EP) 22211421.7

(51) Int. Cl. G05B 19/4155

(52) U.S. Cl. CPC G05B 19/4155; G05B 2219/32287

(57) ABSTRACT

A system produces a machine fluid associated with a machine fluid passport or digital asset and/or a polyisobutene associated with a polyisobutene passport or digital asset. The system includes a chemical production network configured to produce the machine fluid and/or the polyisobutene from one or more input materials through one or more chemical processes associated with one or more environmental attributes. A production operating apparatus is configured to generate the machine fluid and/or polyisobutene passport or digital asset. The chemical production network, or a chemical production system, is configured to provide the machine fluid and/or the polyisobutene in association with the machine fluid and/or the polyisobutene passport or digital asset. Further methods produce the machine fluid and/or the polyisobutene and the passport or digital asset. The machine fluid and the digital asset are provided.

### TECHNICAL FIELD

The present disclosure relates to systems for producing a machine fluid associated with a machine fluid passport or digital asset and/or a polyisobutene associated with a polyisobutene passport or digital asset, methods for producing a machine fluid associated with a machine fluid passport or digital asset and/or a polyisobutene associated with a polyisobutene passport or digital asset, apparatuses for generating a passport or digital asset associated with a machine fluid or associated with a polyisobutene, computer-implemented methods for generating a machine fluid or a polyisobutene passport or digital asset, computer program elements for generating a machine fluid or a polyisobutene passport or digital asset, uses of a machine fluid associated with a machine fluid passport or digital asset and/or a polyisobutene associated with a polyisobutene passport or digital asset, uses of a machine fluid or a polyisobutene passport or digital asset, products produced from the machine fluid associated with the machine fluid passport or digital asset and/or the polyisobutene associated with the polyisobutene passport or digital asset, a machine fluid or a polyisobutene, a machine fluid or polyisobutene passport or digital asset including one or more decentral identifier(s) and data related to environmental impact data, apparatuses for producing a product associated with the machine fluid passport or digital asset and/or with the polyisobutene passport or digital asset and methods for producing a product associated with the machine fluid passport or digital asset and/or with the polyisobutene passport or digital asset.

### TECHNICAL BACKGROUND

In supply chains the environmental impact of each supply chain participants is of great interest. Specifically in the field of chemistry, machine fluids, machine fluid components and polyisobutene are employed for a wide range of applications and are supplied to diverse value chains. In such complex systems transparency between value chain participants is hard to achieve.

### SUMMARY OF THE INVENTION

In one aspect disclosed is a system for producing a machine fluid associated with a machine fluid passport or a digital asset and/or a polyisobutene associated with a polyisobutene passport or a digital asset, the system comprising:
a chemical production network configured to produce the machine fluid and/or the polyisobutene from one or more input material(s) through one or more chemical process(es) of the chemical production network, wherein the one or more input material(s) and/or the one or more chemical process(es) are associated with environmental attribute(s);
a production operating apparatus configured to generate the machine fluid passport or digital asset and/or the polyisobutene passport or digital asset by
   providing a decentral identifier associated with the produced machine fluid and the one or more environmental attribute(s) of the one or more input material(s) and/or the one or more chemical process(s) and/or providing a decentral identifier associated with the produced polyisobutene and the one or more environmental attribute(s) of the one or more input material(s) and/or the one or more chemical process(s);
   linking the decentral identifier and the environmental attribute(s);
the chemical production network or a chemical production system configured to provide the produced machine fluid in association with the machine fluid passport or digital asset and/or to provide the produced polyisobutene in association with the polyisobutene passport or digital asset.

In another aspect disclosed is a system for producing a machine fluid associated with a machine fluid passport and/or a digital asset or a polyisobutene associated with a polyisobutene passport or a digital asset, the system comprising:
a chemical production network configured to produce the machine fluid and/or the polyisobutene from one or more input material(s) through one or more chemical process(es), wherein the one or more input material(s) and/or the one or more chemical process(es) are associated with environmental attribute(s);
a production operating apparatus configured to generate the machine fluid passport or digital asset by providing and/or linking a decentral identifier associated with the produced machine fluid and the one or more environmental attribute(s) associated with the one or more input material(s) and/or the one or more chemical process(es) and/or to generate the polyisobutene passport or digital asset by providing and/or linking a decentral identifier associated with the produced polyisobutene and the one or more environmental attribute(s) associated with the one or more input material(s) and/or the one or more chemical process(es).

In another aspect disclosed is a method for producing a machine fluid associated with a machine fluid passport or a digital asset and/or for producing a polyisobutene associated with a polyisobutene passport or a digital asset, wherein the method comprises:
producing the machine fluid and/or the polyisobutene from one or more input material(s) through one or more chemical process(es) of a chemical production network, wherein the one or more input material(s) and/or the one or more chemical process(es) are associated with environmental attribute(s);
generating the machine fluid passport or digital asset and/or the polyisobutene passport or digital asset by
   providing a decentral identifier associated with the produced machine fluid and one or more environmental attribute(s) associated with the one or more input material(s) and/or the one or more chemical process(es) and/or providing a decentral identifier associated with the produced polyisobutene and the one or more environmental attribute(s) of the one or more input material(s) and/or the one or more chemical process(s);
   linking the decentral identifier and the one or more environmental attribute(s);
providing the produced machine fluid in association with the machine fluid passport or digital asset and/or providing the produced polyisobutene in association with the polyisobutene passport or digital asset.

In another aspect disclosed is a method for producing a machine fluid associated with a machine fluid passport or a digital asset and/or for producing a polyisobutene associated with a polyisobutene passport or a digital asset, wherein the method comprises:
producing the machine fluid and/or the polyisobutene from one or more input material(s) through one or more chemical process(es) of a chemical production network, wherein the one or more input material(s) and/or the one or more chemical process(es) are associated with environmental attribute(s),
generating the machine fluid passport or digital asset by providing and/or linking a decentral identifier associated with the produced machine fluid and one or more environmental attribute(s) of the one or more input material(s) and/or the chemical process(s) and/or generating the polyisobutene passport or digital asset by providing and/or by providing and/or linking a decentral identifier associated with the produced polyisobutene and one or more environmental attribute(s) of the one or more input material(s) and/or the chemical process(s).

In another aspect disclosed is an apparatus for generating a passport or digital asset associated with a machine fluid and/or a passport or digital asset associated with a polyisobutene, wherein the machine fluid and/or the polyisobutene is produced from one or more input material(s) through one or more chemical process(es) of a chemical production network, wherein the one or more input material(s) and/or the one or more chemical process(es) are associated with environmental attribute(s), the apparatus comprising:
a decentral identity provider configured to provide a decentral identifier associated with the produced machine fluid and one or more environmental attribute(s) of the one or more input material(s) and/or the one or more chemical process(es) used to produce the machine fluid and/or to provide a decentral identifier associated with the produced polyisobutene and one or more environmental attribute(s) of the one or more input material(s) and/or the one or more chemical process(es) used to produce the polyisobutene;
an assignor configured link the decentral identifier and the environmental attribute(s);
a passport provider configured to provide the machine fluid passport or digital asset in association with the produced machine fluid and/or to provide the polyisobutene passport or digital asset in association with the produced polyisobutene to a decentral network, wherein the environmental attribute(s) associated with the produced machine fluid is made accessible to a consumer of the produced machine fluid through the machine fluid passport or digital asset and/or wherein the environmental attribute(s) associated with the produced polyisobutene is made accessible to a consumer of the produced polyisobutene through the polyisobutene passport or digital asset.

In another aspect disclosed is a method, e.g. a computer-implemented method, for generating a machine fluid passport or digital asset associated with a machine fluid and/or for generating a polyisobutene passport or digital assed associated with a polyisobutene, wherein the machine fluid and/or the polyisobutene is produced from one or more input material(s) through one or more chemical process(s) of a chemical production network, wherein the one or more input material(s) and/or the one or more chemical process(s) are associated with environmental attribute(s), the method comprising:
providing a decentral identifier associated with the produced machine fluid and one or more environmental attribute(s) of the one or more input material(s) and/or the one or more chemical process(es) used to produce the machine fluid and/or providing a decentral identifier associated with the produced polyisobutene and one or more environmental attribute(s) of the one or more input material(s) and/or the one or more chemical process(es) used to produce the polyisobutene;
linking the decentral identifier and the environmental attribute(s);
providing the machine fluid passport or digital asset in association with the produced machine fluid to a decentral network, wherein the environmental attribute(s) associated with the produced machine fluid is made accessible to a consumer of the produced machine fluid through the machine fluid passport or digital asset and/or providing the polyisobutene passport or digital asset in association with the produced polyisobutene to a decentral network, wherein the environmental attribute(s) associated with the produced polyisobutene is made accessible to a consumer of the produced polyisobutene through the polyisobutene passport or digital asset.

In another aspect disclosed is a computer element, such as a computer readable storage medium, a computer program or a computer program product, comprising instructions, which when executed by a computing node or a computing system, direct the computing node or computing system to carry out the steps of the computer-implemented methods disclosed herein.

In another aspect disclosed is a computer element, such as a computer readable storage medium, a computer program or a computer program product, comprising instructions, which when executed by the apparatuses disclosed herein, direct the apparatuses to carry out steps the apparatuses disclosed herein are configured to execute.

In another aspect disclosed is a machine fluid associated with a machine fluid passport or a digital asset as produced according to the methods disclosed herein. In another aspect disclosed is a polyisobutene associated with a polyisobutene passport or a digital asset as produced according to the methods disclosed herein. In another aspect disclosed a machine fluid associated with a machine fluid passport or a digital asset as produced according to the systems disclosed herein. In another aspect disclosed a polyisobutene associated with a polyisobutene passport or a digital asset as produced according to the systems disclosed herein.

In another aspect disclosed is a machine fluid associated with a machine fluid passport or a digital asset and/or a polyisobutene associated with a polyisobutene passport or digital asset, wherein the machine fluid and/or the polyisobutene is produced from one or more input material(s) through one or more chemical process(es) of a chemical production network, wherein the one or more input material(s) and/or the one or more chemical process(es) are associated with environmental attribute(s), wherein the machine fluid passport or digital asset includes a decentral identifier associated with the produced machine fluid and a link to environmental attribute(s) associated with one or more environmental attribute(s) of one or more input material(s) and/or one or more chemical process(es) used to produce the machine fluid and/or wherein the polyisobutene passport or digital asset includes a decentral identifier associated with the produced polyisobutene and a link to environmental attribute(s) associated with one or more environmental attribute(s) of one or more input material(s) and/or one or more chemical process(es) used to produce the polyisobutene.

In another aspect disclosed is a machine fluid passport as generated according to the methods and/or apparatuses disclosed herein. In another aspect disclosed is a polyisobutene passport or digital asset as generated according to the methods and/or apparatuses disclosed herein.

In another aspect disclosed is a production system for producing a product from the machine fluid associated with the machine fluid passport or digital asset as provided according to the systems, apparatuses or methods disclosed herein. In another aspect disclosed is a production system for producing a product from the polyisobutene associated with the polyisobutene passport or digital asset as provided according to the systems, apparatuses or methods disclosed herein. In another aspect disclosed is a production method for producing a product from the machine fluid associated with the machine fluid passport or digital asset as provided according to the systems, apparatuses or methods disclosed herein. In another aspect disclosed is a production method for producing a product from the polyisobutene associated with the polyisobutene passport or digital asset as provided according to the systems, apparatuses or methods disclosed herein.

In another aspect disclosed is a use of the machine fluid associated with the machine fluid passport or digital asset as disclosed herein for producing a product from the machine fluid associated with the machine fluid passport or digital asset. In another aspect disclosed is a use of the polyisobutene associated with the polyisobutene passport or digital asset as disclosed herein for producing a product from the polyisobutene associated with the polyisobutene passport or digital asset.

In another aspect disclosed is a use of the machine fluid passport or digital asset as disclosed herein for generating a product passport or digital asset associated with a product produced from the machine fluid associated with the machine fluid passport or digital asset. In another aspect disclosed is a use of the polyisobutene passport or digital asset as disclosed herein for generating a product passport or digital asset associated with a product produced from the polyisobutene associated with the polyisobutene passport or digital asset. In another aspect disclosed is a method for using the machine fluid passport or digital asset generated according to the methods disclosed herein in the production of a product produced from the machine fluid associated with the machine fluid passport or digital asset. In another aspect disclosed is a method for using the polyisobutene passport or digital asset generated according to the methods disclosed herein in the production of a product produced from the polyisobutene associated with the polyisobutene passport or digital asset.

In another aspect disclosed is a machine fluid associated with a machine fluid passport or digital asset including a decentral identifier associated with the machine fluid and linked to one or more environmental attribute(s) of the one or more input material(s) and/or the one or more chemical process(es) used to produce the machine fluid. In another aspect disclosed is a polyisobutene associated with a polyisobutene passport or digital asset including a decentral identifier associated with the polyisobutene and linked to one or more environmental attribute(s) of the one or more input material(s) and/or the one or more chemical process(es) used to produce the polyisobutene.

In another aspect disclosed is a use of the machine fluid associated with the machine fluid passport or digital asset for producing a product from the machine fluid and associating the machine fluid passport or digital asset with the product produced from the machine fluid. In another aspect disclosed is a use of the polyisobutene associated with the polyisobutene passport or digital asset for producing a product from the polyisobutene and associating the polyisobutene passport or digital asset with the product produced from the polyisobutene. In another aspect disclosed is a use of the machine fluid associated with the machine fluid passport or digital asset for producing a product from the machine fluid and deriving a product passport or digital asset from the machine fluid passport or digital asset. In another aspect disclosed is a use of the polyisobutene associated with the polyisobutene passport or digital asset for producing a product from the polyisobutene and deriving a product passport or digital asset from the polyisobutene passport or digital asset. In another aspect disclosed is a method for using the machine fluid associated with the digital asset for producing a product from the machine fluid as disclosed herein and deriving a digital asset associated with the product from the machine fluid passport or digital asset. In another aspect disclosed is a method for using the polyisobutene associated with the digital asset for producing a product from the polyisobutene as disclosed herein and deriving a digital asset associated with the product from the polyisobutene passport or digital asset.

Any disclosure and embodiments described herein relate to the methods, the systems, chemical products, machine fluids, machine fluid components, polyisobutene, machine fluid passports or digital assets, polyisobutene passports or digital assets and the computer elements lined out above and below and vice versa. Advantageously, the benefits provided by any of the embodiments and examples equally apply to all other embodiments and examples.

### EMBODIMENTS

In the following, embodiments of the present disclosure will be outlined by ways of embodiments and/or example. It is to be understood that the present disclosure is not limited to said embodiments and/or examples. All terms and definitions used herein are understood broadly and have their general meaning.

The methods, the systems, machine fluids, components of machine fluids, polyisobutene, machine fluid passports or digital assets, polyisobutene passports or digital assets and the computer elements disclosed herein provide an efficient, secure and robust way for sharing or exchanging environmental impact data across different participant nodes in value chains. In particular, by providing machine fluid specific data, machine fluid component specific data and/or polyisobutene specific data via the respective machine fluid and/or polyisobutene passport or digital asset, environmental impacts can be shared and made transparent from the machine fluid and/or the polyisobutene to the product produced from such machine fluid and/or polyisobutene. The machine fluid or polyisobutene passport or digital asset enables secure data exchange, since data access can be controlled by the machine fluid, machine fluid component or polyisobutene provider, e.g. the entity providing the machine fluid or components thereof and/or the polyisobutene. The exchanged data assets can be specific to the machine fluid, machine fluid components and/or polyisobutene as produced and tailored to the needs of the consumer of said products. This way an improved tracking and tracing of machine fluids, machine fluid components and/or polyisobutene can be achieved by securely providing environmental impact data in diverse and highly complex value chains. The environmental impact of machine fluids, machine fluid components and/or polyisobutene can hence be tracked leading to simpler, more efficient and sustainable handling of machine fluids, machine fluid components and/or polyisobutene by value chain participants.

Machine fluids may include any liquid substance commonly used to operate a machine. The substance may be liquid at room temperature under ambient pressure or at temperatures present during operation of the machine. Machine fluids may include components of said machine fluids, such as additives or substances used to prepare said machine fluids. Machines may be stationary machines or movable machines. Stationary machines may include air conditioning devices, power units (nuclear, coal, natural gas, oil, wind, water, solar thermal, geothermal), generators, pumps, hydraulic power units, wind turbines, transformer station, thermal heat pumps, and compressors. Movable machines may include vehicles. Vehicles may include motor vehicles. Examples of motor vehicles may include motorcycles, cars, trucks, buses, vans, minivans, ATV (all-terrain vehicles) and mobility scooters for disabled people). Vehicles may include railed vehicles. Examples of railed vehicles may include trains and trams. Vehicles may include watercraft vehicles. Examples of watercraft vehicles may include ships, boats and underwater vehicles. Vehicles may include amphibious vehicles. Examples of amphibious vehicles may include screw-propelled vehicles and hovercraft. Vehicles may include aircrafts. Examples of aircrafts may include airplanes, helicopters and aerostats. Vehicles may include spacecraft. The stationary or movable machines may be driven by spark-ignited or self-ignition engines, two- or four stroke engines, electrical engines, fuel cells or combinations thereof (hybrid engines).

In an embodiment, the machine fluid associated with the machine fluid passport or digital asset may be selected from the group consisting of lubricants, lubricant additives, lubricant base oils, fuel additives, engine coolants and brake fluids. For instance, the machine fluid may be a lubricant, a lubricant additive, a lubricant base oil, a mixture of lubricant additives, a mixture of lubricant base oils, or a mixture of lubricant additives and lubricant base oils. In another instance, the machine fluid may be a fuel additive or a mixture of fuel additives, e.g. for hydrocarbon-based gasoline or Diesel fuels, which may contain methanol, ethanol or fatty acid alkyl esters. In yet another instance, the machine fluid may be an engine coolant or heat exchanger medium. In yet another instance, the machine fluid may be a brake fluid or hydraulic fluid.

The lubricant is usually a substance capable of reducing friction between surfaces (preferably metal surfaces), such as surfaces of mechanical devices or machines. A mechanical device may be a mechanism consisting of a device that works on mechanical principles, such as the machines previously described. The lubricant is usually a lubricating liquid, lubricating oil or lubricating grease.

The lubricant may be used for various applications such as light, medium and heavy duty engine oils, industrial engine oils, marine engine oils, automotive engine oils, crankshaft oils, compressor oils, refrigerator oils, hydrocarbon compressor oils, very low-temperature lubricating oils and fats, high temperature lubricating oils and fats, wire rope lubricants, textile machine oils, refrigerator oils, aviation and aerospace lubricants, aviation turbine oils, transmission oils, gas turbine oils, spindle oils, spin oils, traction fluids, transmission oils, plastic transmission oils, passenger car transmission oils, truck transmission oils, industrial transmission oils, industrial gear oils, insulat-ing oils, instrument oils, brake fluids, transmission liquids, shock absorber oils, heat distribution medium oils, transformer oils, fats, chain oils, minimum quantity lubricants for metalworking op-erations, oil to the warm and cold working, oil for water-based metalworking liquids, oil for neat oil metalworking fluids, oil for semi-synthetic metalworking fluids, oil for synthetic metalworking fluids, drilling detergents for the soil exploration, hydraulic oils, in biodegradable lubricants or lubricating greases or waxes, chain saw oils, release agents, molding fluids, gun, pistol and rifle lubricants or watch lubricants and food grade approved lubricants.

The lubricant has usually a kinematic viscosity at 40° C. of at least 10, 50, 100, 150, 200, 300, 400, 500, 600, 900, 1400, or 2000 mm²/s. In another form the lubricant has usually a kinematic viscosity at 40° C. from 200 to 30 000 mm²/s (cSt), preferably from 500 to 10 000 mm²/s, and in particular from 1000 to 5000 mm²/s.

The lubricant has usually a kinematic viscosity at 100° C. of at least 2, 3, 5, 10, 20, 30, 40, or 50 mm²/s. In another form the lubricant may have a kinematic viscosity at 100° C. from 10 to 5000 mm²/s (cSt), preferably from 30 to 3000 mm²/s, and in particular from 50 to 2000 mm2/s

The lubricant may have a viscosity index (VI) of at least 150, 160, 170, 180, 190 or 200.

The lubricant may comprise the lubricant base oil and the lubricant additive.

The lubricant may comprise ≥50% to ≤99% by weight or ≥80% to ≤99% by weight or ≥90% to ≤99% by weight of the base oil, related to the total amount of the lubricant.

Suitable lubricant base oils may be selected from the group consisting of mineral oils (Group I, II or III oils), polyalphaolefins (Group IV oils), polymerized and interpolymerized olefins, alkyl naphthalenes, alkylene oxide polymers, silicone oils, phosphate esters and carboxylic acid esters (Group V oils). Definitions for the base oils are the same as those found in the American Petroleum Institute (API) publication “Engine Oil Licensing and Certification System”, Industry Services Department, Fourteenth Edition, December 1996, Addendum 1, December 1998. Said publication categorizes base oils as follows:
a) Group I base oils contain less than 90 percent saturates (ASTM D 2007) and/or greater than 0.03 percent sulfur (ASTM D 2622) and have a viscosity index (ASTM D 2270) greater than or equal to 80 and less than 120.
b) Group II base oils contain greater than or equal to 90 percent saturates and less than or equal to 0.03 percent sulfur and have a viscosity index greater than or equal to 80 and less than 120.
c) Group III base oils contain greater than or equal to 90 percent saturates and less than or equal to 0.03 percent sulfur and have a viscosity index greater than or equal to 120.
d) Group IV base oils contain polyalphaolefins. Polyalphaolefins (PAO) include known PAO materials which typically comprise relatively low molecular weight hydrogenated polymers or oligomers of alphaolefins which include but are not limited to C2 to about C32 alphaolefins with the C8 to about C16 alphaolefins, such as 1-octene, 1-decene, 1-dodecene and the like being preferred. The preferred polyalphaolefins are poly-1-octene, poly-1-decene, and poly-1-dode-cene.
e) Group V base oils contain any base oils not described by Groups I to IV. Examples of Group V base oils include alkyl naphthalenes, alkylene oxide polymers, silicone oils, carboxylic acid esters and phosphate esters.

Synthetic base oils include hydrocarbon oils and halo-substituted hydrocarbon oils such as polymerized and interpolymerized olefins (e.g., polypropylenes, propylene-isobutylene copolymers, chlorinated polybutylenes, poly(1-hexenes), poly(1-octenes), poly(1-decenes)); alkylbenzenes (e.g., dodecylbenzenes, tetradecylbenzenes, dinonylbenzenes, di(2-ethylhexyl)benzenes); polyphenyls (e.g., biphenyls, terphenyls, alkylated polyphenols); and alkylated diphenyl ethers and alkylated diphenyl sulfides and derivative, analogs and homologs thereof.

Alkylene oxide polymers and interpolymers and derivatives thereof where the terminal hydroxyl groups have been modified by esterification, etherification, etc., constitute another class of known synthetic base oils. These are exemplified by polyoxyalkylene polymers prepared by polymeriza-tion of ethylene oxide or propylene oxide, and the alkyl and aryl ethers of polyoxyalkylene poly-mers (e.g., methyl-polyisopropylene glycol ether having a molecular weight of 1000 or diphenyl ether of polyethylene glycol having a molecular weight of 1000 to 1500); and mono- and polycar-boxylic esters thereof, for example, the acetic acid esters, mixed C₃-C₈ fatty acid esters and C₁₃ oxo acid diester of tetraethylene glycol.

Silicon-based oils such as the polyalkyl-, polyaryl-, polyalkoxy- or polyaryloxysilicone oils and sili-cate oils comprise another useful class of synthetic base oils; such base oils include tetraethyl silicate, tetraisopropyl silicate, tetra-(2-ethylhexyl)silicate, tetra-(4-methyl-2-ethylhe-xyl) silicate, tetra-(p-tert-butyl-phenyl) silicate, hexa-(4-methyl-2-ethylhexyl)disiloxane, poly(methyl) siloxanes and poly(methylphenyl)siloxanes. Other synthetic base oils include liquid esters of phosphorous-containing acids (e.g., tricresyl phosphate, trioctyl phosphate, diethyl ester of decylphosphonic acid) and polymeric tetrahydrofurans.

Suitable carboxylic acid esters base oils include the esters of mono and polybasic acids with monoalkanols (simple esters) or with mixtures of mono and polyalkanols (complex esters), and the polyol esters of monocarboxylic acids (simple esters), or mixtures of mono and polycarboxylic acids (complex esters). Esters of the mono/polybasic type include, for example, the esters of monocarboxylic acids such as heptanoic acid, and dicarboxylic acids such as phthalic acid, succinic acid, alkyl succinic acid, alkenyl succinic acid, maleic acid, azelaic acid, suberic acid, sebacic acid, fumaric acid, adipic acid, linoleic acid dimer, malonic acid, alkyl malonic acid, alkenyl malonic acid, etc., with a variety of alcohols such as butyl alcohol, hexyl alcohol, dodecyl alcohol, 2-ethylhexyl alcohol, or mixtures thereof with polyalkanols, etc. Specific examples of these types of esters include nonyl heptanoate, dibutyl adipate, di(2-ethylhexyl) sebacate, di-n-hexyl fumarate, dioctyl sebacate, diisooctyl azelate, diisodecyl azelate, dioctyl phthalate, didecyl phthalate, dieicosyl sebacate, dibutyl-TMP-adipate, etc.

Also suitable carboxylic acid esters base oils can be obtained by reacting one or more polyhydric alcohols, preferably the hindered polyols such as the neopentyl polyols, e.g. neopentyl glycol, trimethylol ethane, 2-methyl-2-propyl-1,3-propanediol, trimethylol propane, trimethylol butane, pentaerythritol and dipentaerythritol with monocarboxylic acids containing at least 4 carbons, normally the C₅ to C₃₀ acids such as saturated straight chain fatty acids including caprylic acid, capric acid, lauric acid, myristic acid, palmitic acid, stearic acid, arachic acid, and behenic acid, or the corresponding branched chain fatty acids or unsaturated fatty acids such as oleic acid, or mixtures thereof, with polycarboxylic acids.

Suitable lubricant additives may be selected from viscosity index improvers, polymeric thickeners, corrosion inhibitors, detergents, dispersants, anti-foam agents, dyes, wear protection additives, extreme pressure additives, anti-wear additives, friction modifiers, metal deactivators, pour point depressants, or demulsifiers.

The total combined amount of the lubricant additive in the lubricant may include ranges of 0-25 wt %, or 0.01-20 wt %, or 0.1-15 wt % or 0.5-10 wt %, or 1-5 wt % of the lubricant.

The viscosity index improvers include high molecular weight polymers that increase the relative viscosity of an oil at high temperatures more than they do at low temperatures. Viscosity index improvers include polyacrylates, polymethacrylates, alkylmethacrylates, vinylpyrrolidone/methacrylate copolymers, poly vinylpyrrolidones, polybutenes, olefin copolymers such as an ethylene-propylene copolymer or a styrene-butadiene copolymer or polyalkene such as PIB, styrene/acrylate copolymers and polyethers, and combinations thereof. The most common VI improvers are methacrylate polymers and copolymers, acrylate polymers, olefin polymers and copolymers, and styrenebutadiene copolymers. Other examples of the viscosity index improver include polymethacrylate, polyisobutylene, alpha-olefin polymers, alpha-olefin copolymers (e.g., an ethylenepropylene copolymer), polyalkylstyrene, phenol condensates, naphthalene condensates, a styrenebutadiene copolymer and the like. Of these, polymethacrylate having a number average molecular weight of 10000 to 300000, and alpha-olefin polymers or alpha-olefin copolymers having a number average molecular weight of 1000 to 30000, particularly ethylene-alpha-olefin copolymers having a number average molecular weight of 1000 to 10000 are preferred. The viscosity index increasing agents can be added and used individually or in the form of mixtures, conveniently in an amount within the range of from ≥0.05 to ≤20.0% by weight, in relation to the weight of the base stock.

Suitable (polymeric) thickeners include, but are not limited to, polyisobutenes (PIB), oligomeric co-polymers (OCPs), polymethacrylates (PMAs), copolymers of styrene and butadiene, or high viscosity esters (complex esters).

Corrosion inhibitors may include various oxygen-, nitrogen-, sulfur-, and phosphorus-containing materials, and may include metal-containing compounds (salts, organometallics, etc.) and nonmetal-containing or ashless materials. Corrosion inhibitors may include, but are not limited to, additive types such as, for example, hydrocarbyl-, aryl-, alkyl-, arylalkyl-, and alkylaryl-versions of detergents (neutral, overbased), sulfonates, phenates, salicylates, alcoholates, carboxylates, salixarates, phosphites, phosphates, thiophosphates, amines, amine salts, amine phosphoric acid salts, amine sulfonic acid salts, alkoxylated amines, etheramines, polyetheramines, amides, imides, azoles, diazoles, triazoles, benzotriazoles, benzothiadoles, mercaptobenzothiazoles, tolyltriazoles (TTZ-type), heterocyclic amines, heterocyclic sulfides, thiazoles, thiadiazoles, mercaptothiadiazoles, dimercaptothiadiazoles (DMTD-type), imidazoles, benzimidazoles, dithiobenzimidazoles, imidazolines, oxazolines, Mannich reactions products, glycidyl ethers, anhydrides, carbamates, thiocarbamates, dithiocarbamates, polyglycols, etc., or mixtures thereof.

Detergents include cleaning agents that adhere to dirt particles, preventing them from attaching to critical surfaces. Detergents may also adhere to the metal surface itself to keep it clean and prevent corrosion from occurring. Detergents include calcium alkylsalicylates, calcium alkylphenates and calcium alkarylsulfonates with alternate metal ions used such as magnesium, barium, or sodium. Examples of the cleaning and dispersing agents which can be used include metal-based detergents such as the neutral and basic alkaline earth metal sulphonates, alkaline earth metal phenates and alkaline earth metal salicylates alkenylsuccinimide and alkenylsuccinimide esters and their borohydrides, phenates, salienius complex detergents and ashless dispersing agents which have been modified with sulphur compounds. These agents can be added and used individually or in the form of mixtures, conveniently in an amount within the range of from ≥0.01 to ≤1.0% by weight in relation to the weight of the base stock; these can also be high total base number (TBN), low TBN, or mixtures of high/low TBN.

Dispersants are lubricant additives that help to prevent sludge, varnish and other deposits from forming on critical surfaces. The dispersant may be a succinimide dispersant (for example N-substituted long chain alkenyl succinimides), a Mannich dispersant, an ester-containing dispersant, a condensation product of a fatty hydrocarbyl monocarboxylic acylating agent with an amine or ammonia, an alkyl amino phenol dispersant, a hydrocarbyl-amine dispersant, a polyether dispersant or a polyetheramine dispersant. In one embodiment, the succinimide dispersant includes a polyisobutylene-substituted succinimide, wherein the polyisobutylene from which the dispersant is derived may have a number average molecular weight of about 400 to about 5000, or of about 950 to about 1600. In one embodiment, the dispersant includes a borated dispersant. Typically, the borated dispersant includes a succinimide dispersant including a polyisobutylene succinimide, wherein the polyisobutylene from which the dispersant is derived may have a number average molecular weight of about 400 to about 5000. Borated dispersants are described in more detail above within the extreme pressure agent description.

Anti-foam agents may be selected from silicones, polyacrylates, and the like. The amount of anti-foam agent in the lubricant compositions described herein may range from ≥0.001 wt.-% to ≤0.1 wt.-% based on the total weight of the formulation. As a further example, an anti-foam agent may be present in an amount from about 0.004 wt.-% to about 0.008 wt.-%.

Suitable extreme pressure agent is a sulfur-containing compound. In one embodiment, the sulfur-containing compound may be a sulfurised olefin, a polysulfide, or mixtures thereof. Examples of the sulfurised olefin include a sulfurised olefin derived from propylene, isobutylene, pentene; an organic sulfide and/or polysulfide including benzyldisulfide; bis-(chlorobenzyl) disulfide; dibutyl tetrasulfide; di-tertiary butyl polysulfide; and sulfurised methyl ester of oleic acid, a sulfurised alkylphenol, a sulfurised dipentene, a sulfurised terpene, a sulfurised Diels-Alder adduct, an alkyl sulphenyl N′N-dialkyl dithiocarbamates; or mixtures thereof. In one embodiment, the sulfurised olefin includes a sulfurised olefin derived from propylene, isobutylene, pentene or mixtures thereof. In one embodiment the extreme pressure additive sulfur-containing compound includes a dimercaptothiadiazole or derivative, or mixtures thereof. Examples of the dimercaptothiadiazole include compounds such as 2,5-dimercapto-1,3,4-thiadiazole or a hydrocarbyl-substituted 2,5-dimercapto-1,3,4-thiadiazole, or oligomers thereof. The oligomers of hydrocarbyl-substituted 2,5-dimercapto-1,3,4-thiadiazole typically form by forming a sulfur-sulfur bond between 2,5-dimercapto-1,3,4-thiadiazole units to form derivatives or oligomers of two or more of said thiadiazole units. Suitable 2,5-dimercapto-1,3,4-thiadiazole derived compounds include for example 2,5-bis(tert-nonyldithio)-1,3,4-thiadiazole or 2-tert-nonyldithio-5-mercapto-1,3,4-thiadiazole. The number of carbon atoms on the hydrocarbyl substituents of the hydrocarbyl-substituted 2,5-dimercapto-1,3,4-thiadiazole typically include 1 to 30, or 2 to 20, or 3 to 16. Extreme pressure additives include compounds containing boron and/or sulfur and/or phosphorus. The extreme pressure agent may be present in the lubricant compositions at 0 wt.-% to about 20 wt.-%, or at about 0.05 wt.-% to about 10.0 wt.-%, or at about 0.1 wt.-% to about 8 wt.-% of the lubricant composition.

Examples of anti-wear additives include organo borates, organo phosphites such as didodecyl phosphite, organic sulfur-containing compounds such as sulfurized sperm oil or sulfurized terpenes, zinc dialkyl dithiophosphates, zinc diaryl dithiophosphates, phosphosulfurized hydrocarbons and any combinations thereof.

Friction modifiers may include metal-containing compounds or materials as well as ashless compounds or materials, or mixtures thereof. Metal-containing friction modifiers include metal salts or metal-ligand complexes where the metals may include alkali, alkaline earth, or transition group metals. Such metal-containing friction modifiers may also have low-ash characteristics. Transition metals may include Mo, Sb, Sn, Fe, Cu, Zn, and others. Ligands may include hydrocarbyl derivative of alcohols, polyols, glycerols, partial ester glycerols, thiols, carboxylates, carbamates, thiocarbamates, dithiocarbamates, phosphates, thiophosphates, dithiophosphates, amides, imides, amines, thiazoles, thiadiazoles, dithiazoles, diazoles, triazoles, and other polar molecular functional groups containing effective amounts of O, N, S, or P, individually or in combination. In particular, Mo-containing compounds can be particularly effective such as for example Mo-dithiocarbamates, Mo(DTC), Mo-dithiophosphates, Mo(DTP), Mo-amines, Mo (Am), Mo-alcoholates, Mo-alcohol-amides, and the like.

Ashless friction modifiers may also include lubricant materials that contain effective amounts of polar groups, for example, hydroxyl-containing hydrocarbyl base oils, glycerides, partial glycerides, glyceride derivatives, and the like. Polar groups in friction modifiers may include hydrocarbyl groups containing effective amounts of O, N, S, or P, individually or in combination. Other friction modifiers that may be particularly effective include, for example, salts (both ash-containing and ashless derivatives) of fatty acids, fatty alcohols, fatty amides, fatty esters, hydroxyl-containing carboxylates, and comparable synthetic long-chain hydrocarbyl acids, alcohols, amides, esters, hydroxy carboxylates, and the like. In some instances, fatty organic acids, fatty amines, and sulfurized fatty acids may be used as suitable friction modifiers. Examples of friction modifiers include fatty acid esters and amides, organo molybdenum compounds, molybdenum dialkylthiocarbamates and molybdenum dialkyl dithiophosphates.

Suitable metal deactivators include benzotriazoles and derivatives thereof, for example 4- or 5-alkylbenzotriazoles (e.g. triazole) and derivatives thereof, 4,5,6,7-tetrahydrobenzotriazole and 5,5′-methylenebisbenzotriazole; Mannich bases of benzotriazole or triazole, e.g. 1-[bis(2-ethyl-hexyl) aminomethyl) triazole and 1-[bis(2-ethylhexyl) aminomethyl)benzotriazole; and alkoxy-alkylbenzotriazoles such as 1-(nonyloxymethyl)benzotriazole, 1-(1-butoxyethyl) benzotriazole and 1-(1-cyclohexyloxybutyl) triazole, and combinations thereof. Additional non-limiting examples of the one or more metal deactivators include 1,2,4-triazoles and derivatives thereof, for example 3-alkyl(or aryl)-1, 2,4-triazoles, and Mannich bases of 1,2,4-triazoles, such as 1-[bis(2-ethylhexyl) aminomethy1-1, 2,4-triazole; alkoxyalky1-1, 2,4-triazoles such as 1-(1-bu-tox-yethyl)-1, 2,4-triazole; and acylated 3-amino-1, 2,4-triazoles, imidazole derivatives, for example 4,4′-methylenebis(2-undecyl-5-methylimidazole) and bis[(N-methyl)imidazol-2-yl]-carbinol octyl ether, and combinations thereof. Further non-limiting examples of the one or more metal deactivators include sulfur-containing heterocyclic compounds, for example 2-mercapto-benzothiazole, 2,5-dimercapto-1, 3,4-thia-diazole and derivatives thereof; and 3,5-bis[di(2-ethylhexyl) aminomethyl]-1, 3,4-thiadiazolin-2-one, and combinations thereof. Even further non-limiting examples of the one or more metal deactivators include amino compounds, for example salicylidenepropylenediamine, salicylami-noguanidine and salts thereof, and combinations thereof. The one or more metal deactivators are not particularly limited in amount in the composition but are typically present in an amount of from about 0.01 to about 0.1, from about 0.05 to about 0.01, or from about 0.07 to about 0.1, wt.-% based on the weight of the composition. Alternatively, the one or more metal deactivators may be present in amounts of less than about 0.1, of less than about 0.7, or less than about 0.5, wt.-% based on the weight of the composition.

Pour point depressants (PPD) include polymethacrylates, alkylated naphthalene derivatives, and combinations thereof. Commonly used additives such as alkylaromatic polymers and polymethacrylates are also useful for this purpose. Typically, the treat rates range from ≥0.001 wt.-% to ≤1.0 wt.-%, in relation to the weight of the base stock.

Demulsifiers include trialkyl phosphates, and various polymers and copolymers of ethylene glycol, ethylene oxide, propylene oxide, or mixtures thereof.

Fuel Additives often comprise quaternized nitrogen compounds which are described in
WO 2006/135881 A1, page 5, line 13 to page 12, line 14;
WO 10/132259 A1, page 3, line 28 to page 10, line 25;
WO 2008/060888 A2, page 6, line 15 to page 14, line 29;
WO 2011/095819 A1, page 4, line 5 to page 9, line 29;
GB 2496514 A, paragraph [00012] to paragraph [00041];
WO 2013/117616 A1, page 3, line 34 to page 11, line 2;
WO 14/202425 A2, page 3, line 14 to page 5, line 9;
WO 14/195464 A1, page 15, line 31 to page 45, line 26 and page 75, lines 1 to 4;
WO 15/040147 A1, page 4, line 34 to page 5, line 18 and page 19, line 11 to page 50, line 10;
WO 14/064151 A1, page 5, line 14 to page 6, line 17 and page 16, line 10 to page 18, line 12;
WO 2013/064689 A1, page 18, line 16 to page 29, line 8;
WO 2013/087701 A1, page 13, line 25 to page 19, line 30,
WO 13/000997 A1, page 17, line 4 to page 25, line 3, and
WO 12/004300, page 5, lines 20 to 30, page 8, line 1 to page 10, line 10, and page 19, line 29 to page 28, line 3,

each of which is incorporated herein by reference.

Polyisobutylene amine as gasoline additive for e.g. valve cleaning is based on high-reactivity polyisobutene (after hydroformylation and amination with ammonia), molecular weight Mn=1000.

Engine coolants are usually aqueous compositions of alkylene glycols, preferably mono ethylene glycol, further comprising other additives, such as corrosion inhibitors, defoamers, bittering agents, and colourants.

Brake fluids and hydraulic fluids may be hydrocarbon based or comprise mixtures of alkyl oligo alkylene glycol borates with oligo alkylene glycols, further comprising other additives, such as corrosion inhibitors and defoamers.

The polyisobutene may be selected from low, medium or high molecular polyisobutene having a number average molecular weight Mn from 500 to 1,000,000 g/mol measured by gel permeation chromatography. Low molecular weight polyisobutene, e.g. exhibiting an Mn of from 500 to 2500, preferably from 550 to 2300, more preferably from 750 to 1300, even more preferably from 950 to 105 g/mol, further may be highly reactive, e.g. with a content of terminal double bonds of at least 50%, preferably at least 70%, more preferably at least 75%, even more pref at least 80%, and especially at least 85%.

High molecular weight polyisobutene is preferably obtainable by a process as described in WO 15/095960 which is incorporated herein by reference.

The chemical production network may include one or more one or more chemical and/or mechanical process(es). The chemical production network may produce one or more output material(s) through chemical and/or mechanical processing. The chemical production network may include multiple types of production processes for producing one or more output material(s) from one or more input material(s). The chemical production network may produce one or more output material(s) from input material(s) provided to the chemical production network. The chemical production network may include a complex production network producing multiple chemical products via multiple production process(es). The chemical production network may include connected, interconnected and/or non-connected production process(es). The chemical production network may include a composite or Verbund network.

The chemical production network may include identity preserving or segregated production process(es). Identity preserving or segregated in this context may refer to environmental attribute(s) of input material(s) being preserved or segregated in the production process(es). Examples are non-fossil, e.g. renewable or recycled, input materials used to produce the one or more machine fluid(s) or polyisobutene without fossil content. Further examples are fossil input material(s) used to produce the one or more machine fluid(s) or polyisobutene with fossil content. Chemical production networks may include non-identity preserving or non-segregated production process(es). Non-identity preserving or non-segregated in this context may refer to non-fossil input material(s) being mixed with fossil input material(s) to produce the machine fluid(s) or polyisobutene. For example, fossil and renewable input materials may be mixed to produce the machine fluid(s) or polyisobutene with fossil and renewable content.

The chemical production network may include one or more production process(es) with multiple production steps. The production steps included in the chemical network may be defined by the physical system boundary of the chemical production network. The system boundary may be defined by location and/or control over production processes or steps. The system boundary may be defined by a site of the chemical production network. The system boundary may be defined by production process(es) or step(s) controlled by one entity or multiple entities jointly. The system boundary may be defined by the value chain with staggered production process(es) or step(s) to the chemical end-product, which may be controlled by multiple entities jointly or separately. The chemical production network may include a waste collection, a sorting step, a recycling step such as pyrolysis, a cracking step such as steam cracking, a separation step to separate intermediates of one process step and further processing steps to convert such intermediates to output material(s), in particular the produced machine fluid(s) or the produced polyisobutene leaving the system boundary of the chemical production network. The input material(s) may enter the physical system boundary of the chemical production network. The entry point(s) of the chemical production network may be marked by the entry of input material(s) to the chemical production network or the system boundary of the chemical network. The output material(s), in particular the produced machine fluid(s) or the produced polyisobutene, may leave the physical system boundary of the chemical production network. The exit point(s) of the chemical production network may be marked by the exit of output material(s), in particular the produced machine fluid(s) or the produced polyisobutene, from the chemical production network or the system boundary of the chemical network.

The chemical production network may include one or more production chain(s) for the production of machine fluid(s) and/or polyisobutene. The production chain(s) for the production of machine fluids and/or polyisobutene may be interconnected. The production chain(s) for the production of machine fluid(s) and/or polyisobutene may be interconnected with production chain(s) for the production of other output material(s). The production chain(s) for the production of machine fluid(s) and/or polyisobutene may include production chain(s) for the production of intermediates used to produce machine fluid(s) and/or polyisobutene. The production chain(s) for the production of machine fluid(s) and/or polyisobutene may use input material(s) provided by chemical network(s) for the production of intermediates usable to produce machine fluid(s) and/or polyisobutene.

One or more input material(s) may be provided to the chemical production network for one or more output material(s), in particular machine fluid(s) and/or polyisobutene. The output material(s), in particular machine fluid(s) and/or polyisobutene, may be produced from one or more input material(s) through one or more chemical process(s) of the chemical production network. The input material may comprise any input material entering the chemical production network at any entry point. The input material may include organic or inorganic input material(s). The input material may be a pre-cursor product, an intermediate material, a feedstock or a raw material used to produce one or more output material(s), in particular machine fluid(s) and/or polyisobutene. The input material may be fed to the chemical production network to produce one or more output material(s), in particular machine fluid(s) and/or polyisobutene. The input material may be fed to chemical production network including one or more production process(es) with multiple process steps. The input material may be fed to the chemical production network at the start of the production process or at any intermediate stage of the production process. The input materials entering the chemical production network may be used to produce one or more output material(s), in particular machine fluid(s) and/or polyisobutene.

The input material may be associated with an input material identifier. The input material identifier may comprise any identifier uniquely associated with the input material. The input material identifier may be associated with the physical entity of the input material. The input material identifier may be associated with a single batch of input material. The input material identifier may be associated with a group of input materials. The identifier may be associated with multiple physical entities of the input material. The input material identifier may be associated with continuous or semi-continuous stream of input material. The input material identifier may be associated with a stream of the input material e.g. over a certain time period or from a certain supplier. The input material identifier may be linked or connected to one or more environmental attribute(s).

Environmental attribute may refer to a property related to the environmental impact. Such property may be the property of input material(s), chemical process(es), chemical production network(s) and/or machine fluid(s) and/or polyisobutene. The environmental attribute may indicate an environmental performance of input material(s), chemical process(es), chemical production network(s) and/or machine fluid(s) and/or polyisobutene. The environmental attribute may be derived from properties of input material(s), chemical process(es), chemical production network(s) and/or machine fluid(s) and/or polyisobutene. The environmental attribute may be associated with the environmental impact of input material(s), chemical process(es), chemical production network(s) and/or machine fluid(s) and/or polyisobutene at any stage of the lifecycle of the input material(s) and/or the machine fluid(s) and/or the polyisobutene. The stages may include providing raw material, providing feedstock, producing chemical products, such as intermediate products or end products, producing discrete products by using the chemical products, using chemical products or discrete products, treating end-of-life products, recycling end-of-life products, disposing end-of-life products, reusing components from end-of-life products or any subset of stages. The environmental attribute may be specified or may be derived from any activity of one or more entities participating at any stage of the lifecycle of one or more material(s) or product(s).

The environmental attribute may include one or more characteristic(s) that are attributable to environmental impact of input material(s), chemical process(es), chemical production network(s) and/or the machine fluid(s) and/or the polyisobutene. The environmental attribute may include environmental, technical, recyclability or circularity characteristics(s) associated with the environmental impact of input material(s), chemical process(es), chemical production network(s) and/or machine fluid(s) and/or polyisobutene.

The environmental attribute may include one or more characteristic(s) that are attributable to the environmental impact of input material(s), chemical process(es), chemical production network(s) and/or machine fluid(s) and/or polyisobutene. The environmental attribute may include environmental, technical, recyclability or circularity characteristics(s) associated with the environmental impact of input material(s), chemical process(es), chemical production network(s) and/or machine fluid(s) and/or polyisobutene. The one or more environmental attribute(s) may be attributable to the environmental impact of the machine fluid(s) and/or the polyisobutene. The one or more environmental attribute(s) may relate to environmental, technical, recyclability, circularity and/or complementary risk characteristic(s) of the machine fluid(s) and/or the polyisobutene.

Environmental characteristic(s) may specify or quantify ecological criteria associated with environmental impact. Environmental characteristic(s) may be or may be derived from measurements taken during the lifecycle. Environmental characteristics may be determined at any stage of the lifecycle and may characterize the environmental impact for such stage or up to such stage. Environmental characteristic(s) may for example include carbon footprint, greenhouse gas emissions, resource usage, air emissions, ozone depletion potential, water pollution, noise pollution, energy consumption, waste reduction, or eutrophication potential. Environmental characteristic(s) may for example include product characteristics related to the production of the product like bio based, vegetable based, animal based, halogen-free, fluorine-free, vegan, halal, kosher, palm oil-free, natural, tox-fee, volatile organic compounds-free or any combinations thereof.

Technical characteristic(s) may specify or quantify performance at least indirectly associated with the environmental impact. Technical characteristic(s) may be or may be derived from measurements taken during the lifecycle. Technical characteristics may be determined at any stage of the lifecycle and may characterize the performance for such stage or up to such stage. Technical characteristic(s) may for example include chemical composition data, raw material composition such as bio-based or recycled input material content specifying e.g. x % non-fossil and y % fossil content, bill of materials, product specification data such as product purity, product form (as indication to their impact on dust formation/release), safety data, product extractability, migration data, toxicological data or ecotoxicological data, product component data, safety data, application property data, application instructions, quality data or any combinations thereof.

Circularity characteristic(s) may specify or quantify the life cycle characteristics associated with circular uses. Circularity characteristic(s) may be or may be derived from measurements taken during the lifecycle. Circularity characteristic(s) may be or may be derived from circular data recorded in one or more prior lifecycle(s) including reuse. Circularity characteristic(s) may be determined at any stage of the lifecycle and may characterize the reuse or recycling performance for such stage or up to such stage. Circularity characteristic(s) may for example include recycling data, reuse rate, recycling rate, recycling loops, reuse performance, reused quality or any combinations thereof.

Recyclability characteristic(s) may specify or quantify life cycle characteristics associated with recycling uses. Recyclability characteristic(s) may include the composition of the material including specifically tailored constituents making the material suitable for recycling. Recyclability characteristic(s) may be or may be derived from measurements taken during the lifecycle. Recyclability characteristic(s) may be or may be derived from recycling data recorded in one or more prior lifecycle(s). Recyclability characteristics may be determined at any stage of the lifecycle and may characterize the recycling performance for such stage or up to such stage. Recyclability characteristic(s) may for example include recycling data, number of reuses, recyclate composition, recyclate quality, waste stream composition, waste stream quality or any combinations thereof.

In one embodiment the machine fluid passport or digital asset associated with the machine fluid(s) may include mass balanced environmental attributes related to the input material. In one embodiment the polyisobutene passport or digital asset associated with the polyisobutene may include mass balanced environmental attributes related to the input material. Mass balanced environmental attributes may include environmental attributes of the input material(s) used to produce the machine fluid(s) and/or the polyisobutene, which are tracked and by mass attributable to the machine fluid(s) and/or the polyisobutene. The environmental impact of input material(s) may be determined based on input material(s) used in the chemical process(s) to produce the machine fluid(s) and/or the polyisobutene. For example, bio-based, renewable and/or recycled content of input material(s) used to produce the machine fluid(s) and/or polyisobutene may be tracked. Further for example, properties of the chemical process(es) used to produce the machine fluid(s) and/or polyisobutene may be tracked. Examples of tracked process properties related to the environmental impact include water consumption, CO₂ emissions and/or Greenhouse Gas (GHG) emissions, amount of waste generation, mixed material generation, design for recycling, energy consumption, processing properties such as less waste or less loss of properties. The properties may be tracked based on a certificate from a certifying agency. The properties may be tracked based on inherent physical properties derived from measurements.

In one embodiment the produced machine fluid(s) is/are connected to the decentral identifier physically identifying the produced machine fluid(s). In one embodiment the produced polyisobutene is connected to the decentral identifier physically identifying the produced polyisobutene. The production operating apparatus may be configured to provide the decentral identifier associated with a physical entity of the produced machine fluid(s) and/or the produced polyisobutene. The production operating apparatus may be configured to link the decentral identifier to a physical identifier of the produced machine fluid(s) and/or the produced polyisobutene. The production operating apparatus may be configured to assign the decentral identifier to the physical identifier connected to the produced machine fluid(s) and/or the produced polyisobutene. The production operating apparatus may be configured to assign the decentral identifier to the physical identifier physically connected to the produced machine fluid(s) and/or the produced polyisobutene.

In one embodiment the decentral identifier relates to data associated with at least one product produced from the machine fluid(s), wherein the one or more environmental attribute(s) associated with the at least one product is derived from one or more environmental attribute(s) associated with the machine fluid(s). In one embodiment the decentral identifier relates to data associated with at least one product produced from the polyisobutene, wherein the one or more environmental attribute(s) associated with the at least one product is derived from one or more environmental attribute(s) associated with the polyisobutene. The one or more environmental attribute(s) associated with the machine fluid(s) or the polyisobutene may be associated with the one or more input material(s) and/or the chemical process(es) used to produce the machine fluid(s) and/or the polyisobutene. The decentral identifier may relate to any identifier uniquely associated with the machine fluid(s) or the polyisobutene. The decentral identifier may be associated with the physical entity of the machine fluid(s) or the polyisobutene. The decentral identifier may refer to a single batch of the produced machine fluid(s) or the produced polyisobutene. The decentral identifier may be associated with a group of machine fluid(s) or polyisobutene. The identifier may refer to multiple physical entities of the produced machine fluid(s) or the produced polyisobutene. The decentral identifier may be associated with continuous or semi-continuous stream of machine fluid(s) or polyisobutene. The identifier may refer to a stream of the machine fluid(s) or polyisobutene e.g. over a certain time period or from a certain supplier.

In one embodiment the one or more environmental attribute(s) associated with the machine fluid(s) are provided from at least one balancing account configured to store environmental attribute(s) associated with input material(s). In one embodiment the one or more environmental attribute(s) associated with the polyisobutene are provided from at least one balancing account configured to store environmental attribute(s) associated with input material(s). The balancing account may relate to storage structure associated with metadata, such as an environmental attribute type. For instance, the balancing account may be associated with metadata indicating the environmental attribute type to be recycled-content or bio-based. An inbound allocator may be configured to allocate the one or more environmental attribute(s) associated with input material(s) to at least one balancing account e.g. on entry of the input material to the chemical production network. The balancing account may be associated with the respective environmental attribute type. An outbound assigner may be configured to assign at least one environmental attribute from the at least one balancing account associated with the respective environmental attribute to the decentral identifier. One or more environmental attribute(s) may be assigned to the at least one decentral identifier. Assignment may include de-allocation of the one or more environmental attributes from the balancing account associated with the respective environmental attribute type. By using the balancing accounts environmental attributes of input materials can be reliably tracked and assigned to produced machine fluid(s) and/or produced polyisobutene.

An inbound allocator may be configured to allocate the one or more environmental attribute(s) to at least one balancing account associated with the respective environmental attribute, e.g. on input material entering the chemical production network. The balancing account may be associated with the respective environmental attribute type. The one or more environmental attribute(s) associated with the input materials may be allocated to the balancing account associated with the respective environmental attribute type. An outbound assigner may be configured to assign or link at least one environmental attribute from the at least one balancing account associated with the respective environmental attribute type to the decentral identifier. This may include de-allocation of the one or more environmental attribute(es) from the balancing account associated with the respective environmental attribute type. By using the balancing accounts environmental attributes can be tacked through the chemical production network. This way the environmental attributes may be detached from the material flow. Attribution of environmental attribute(s) may be conducted on a mass balance basis for the chemical production network. In such approach the total mass of input materials and produced machine fluid(s) or produced polyisobutene as well as the attribution of respective environmental attribute(s) associated with input materials and produced machine fluid(s) or produced polyisobutene are balanced.

In one embodiment the one or more environmental attribute(s) are associated with at least one property related to the environmental impact of the one or more input material(s) and/or the chemical process(es). The one or more environmental attribute(s) may specify environmental properties of the input material(s) used to produce the machine fluid(s) and/or the polyisobutene and/or the one or more environmental attribute(s) may specify environmental properties of the chemical process(es) used to produce the machine fluid(s) and/or the polyisobutene. The one or more environmental attribute(s) may be generated from environmental properties of the input material(s) used to produce the machine fluid(s) and/or the polyisobutene, process data associated with the chemical processing of the input material(s) and/or energy data associated with the energy consumption of the chemical processing. The one or more environmental attribute(s) may include a recycled content associated with the input material(s) and allocated or allocatable to the produced machine fluid(s) and/or the produced polyisobutene, a renewable content associated with the input material(s) and allocated or allocatable to the produced machine fluid(s) and/or the produced polyisobutene, and/or a product carbon footprint associated with the produced machine fluid(s) and/or the produced polyisobutene.

In one embodiment the production operating apparatus is configured to gather environmental attributes associated with the produced machine fluid(s) and/or the produced polyisobutene before, during and/or after production of the machine fluid(s) and/or the polyisobutene by the chemical production network. The environmental attributes associated with the produced machine fluid(s) and/or the produced polyisobutene may relate to input material(s). The environmental attributes associated with input materials may be provided before, during and/or after production of the machine fluid(s) and/or the polyisobutene by the chemical production network. The environmental attributes associated with input materials may be allocated to at least one balancing account before, during and/or after production of the machine fluid(s) and/or the polyisobutene by the chemical production network. The environmental attributes associated with the produced machine fluid(s) and/or the produced polyisobutene may relate to environmental properties generated from process data associated with the chemical processing of the input material(s) and/or energy data associated with the energy consumption of the chemical processing. The environmental attributes associated with the produced machine fluid(s) and/or the produced polyisobutene may be generated before, during and/or after production of the machine fluid(s) and/or the polyisobutene by the chemical production network. The process data associated with the chemical processing of the input material(s) and/or energy data associated with the energy consumption of the chemical processing may be gathered prior, during and/or after production of the machine fluid(s) and/or the polyisobutene.

In one embodiment the machine fluid passport or the digital asset may include the decentral identifier associated with the machine fluid and the one or more environmental attribute(s) linked to the decentral identifier. In one embodiment the polyisobutene passport or the digital asset may include the decentral identifier associated with the polyisobutene and the one or more environmental attribute(s) linked to the decentral identifier. The one or more environmental attribute(s) may be linked to the decentral identifier included in the machine fluid passport or the digital asset or included in the polyisobutene passport or digital asset. The one or more environmental attribute(s) may be stored in a data base associated with or of the machine fluid(s) and/or polyisobutene producer for access by any consumer of the produced machine fluid(s) and/or the produced polyisobutene. The one or more environmental attribute(s) may be stored in a data base associated with or of the machine fluid(s) and/or polyisobutene producer for transfer to a consumer of the produced machine fluid(s) and/or the produced polyisobutene e.g. when accessed or on providing the machine fluid(s) and/or the polyisobutene. The decentral identifier may comprise any unique identifier uniquely associated with the machine fluid(s) and/or polyisobutene producer and machine fluid and/or polyisobutene data such as the environmental attributes. The decentral identifier may include on or more Universally Unique Identifier(s) (UUID(s)) or a Digital Identifier(s) (DID(s)). The decentral identifier may be issued by a central or decentral identity issuer. The decentral identifier may be linked to authentication and/or authorization information. Via the decentral identifier and its unique association with the machine fluid(s) and/or polyisobutene producer and machine fluid and/or polyisobutene data, such as the environmental attributes, access to the machine fluid and/or the polyisobutene data may be controlled by the machine fluid and/or polyisobutene producer. This contrasts with central authority schemes, where identifiers are provided by such central authority and access to data is controlled by such central authority. Decentral in this context refers to the usage of the identifier in implementation as controlled by the data owner, such as the machine fluid and/or polyisobutene producer.

The decentral identifier may be uniquely associated with the machine fluid or the polyisobutene or the physical entity of the machine fluid or the polyisobutene, e.g. as packaged for transportation to the consumer of the machine fluid and/or the polyisobutene. The decentral identifier may be uniquely to the one or more environmental attribute(s). The machine fluid passport or the digital asset may include one or more digital representation(s) pointing to machine fluid data including the environmental attribute(s) or parts thereof including the environmental attribute(s). The polyisobutene passport or the digital asset may include one or more digital representation(s) pointing to polyisobutene data including the environmental attribute(s) or parts thereof including the environmental attribute(s). The digital representation may comprise at least one interface to a data providing service. It may further include at least one interface to a data consuming service. It may include an endpoint for data exchange or sharing (resource endpoint) or an endpoint for service interaction (service endpoint), that is uniquely identified via a communication protocol. The digital representation(s) pointing to machine fluid data or parts thereof may be uniquely associated with the decentral identifier. The digital representation(s) pointing to polyisobutene data or parts thereof may be uniquely associated with the decentral identifier.

The machine fluid passport or the digital asset may comprise or be connected to a digital representation of machine fluid data, such as environmental attribute(s). The polyisobutene passport or the digital asset may comprise or be connected to a digital representation of polyisobutene data, such as environmental attribute(s). The digital representation may include a representation for accessing the machine fluid or polyisobutene data, such as environmental attribute(s) or part thereof. The digital representation may include a representation of machine fluid or polyisobutene data, such as environmental attribute(s). The machine fluid passport or the digital asset may include or be connected to data related to the machine fluid data, such as environmental attribute(s), the authentication information and the decentral identifier. The polyisobutene passport or the digital asset may include or be connected to data related to the polyisobutene data, such as environmental attribute(s), the authentication information and the decentral identifier. The data related to the machine fluid data, such as environmental attribute(s), may include the digital representation of the machine fluid data, such as environmental attribute(s). The data related to the polyisobutene data, such as environmental attribute(s), may include the digital representation of the polyisobutene data, such as environmental attribute(s).

### BRIEF DESCRIPTION OF THE DRAWINGS

In the following, the present disclosure is further described with reference to the enclosed figures:

FIG. 1 illustrates schematically an example of a chemical production network producing one or more output material(s) from one or more input material(s) in connection with a production operating system including an attribute management system.

FIG. 2 illustrates schematically an example of attributing environmental attributes of input materials to output materials of the chemical production network.

FIG. 3 illustrates schematically an example of attributing environmental attributes of input materials and chemical processes to an output material of the chemical production network.

FIG. 4 illustrates schematically another example of a method or apparatus for providing environmental attributes associated with output materials to a material user as data consumer via a decentral network.

FIG. 5 illustrates schematically an example of a method or apparatus for providing environmental attributes of output materials across value chains via the decentral network.

FIG. 6A illustrates schematically an example of a chemical production network for producing a lubricant base oil associated with a machine fluid passport or digital asset.

FIG. 6B illustrates schematically an example of a chemical production network for producing polyisobutene(s) associated with a polyisobutene passport or digital asset.

FIG. 6C illustrates schematically an example of a chemical production network for producing fuel additive(s) associated with a machine fluid passport or digital asset.

FIG. 6D illustrates schematically an example of a chemical production network for producing engine coolant(s) associated with a machine fluid passport or digital asset.

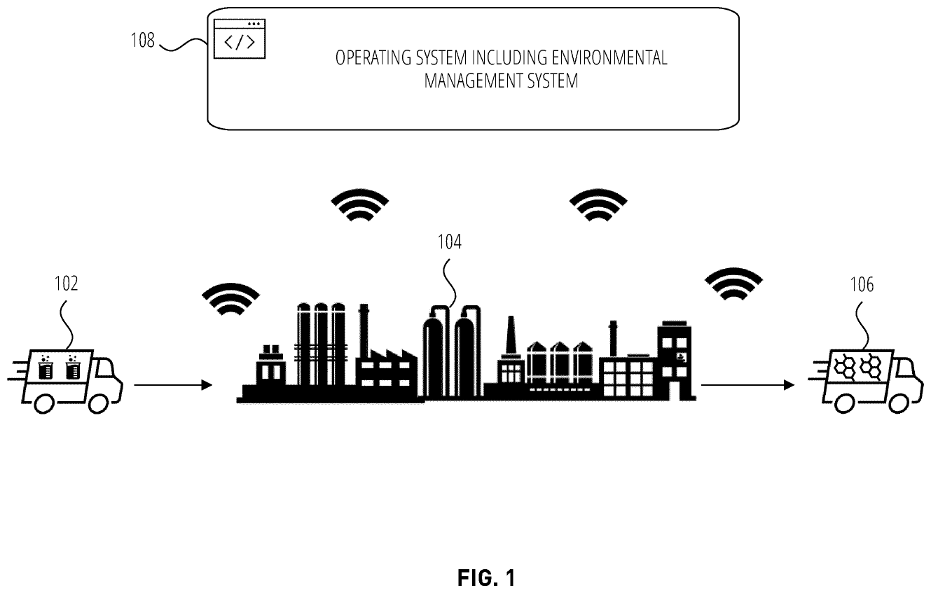

FIG. 1 illustrates an example of a chemical production network 104 producing one or more output material(s), in particular machine fluid(s) and/or polyisobutene(s), 106 from one or more input material(s) 102 in connection with a production operating system 108 including an attribute management system. The machine fluid(s) may be selected from the group consisting of lubricants, lubricant additives, lubricant base oils, fuel additives, engine coolants and brake fluids. For instance, the machine fluid may be a lubricant, a lubricant additive, a lubricant base oil, a mixture of lubricant additives, a mixture of lubricant base oils, or a mixture of lubricant additives and lubricant base oils. In another instance, the machine fluid may be a fuel additive or a mixture of fuel additives. In yet another instance, the machine fluid may be an engine coolant. In yet another instance, the machine fluid may be a brake fluid.

For producing one or more output material(s), in particular machine fluid(s) and/or polyisobutene(s), 106 different input materials 102 may be provided as physical inputs to the chemical production network 104. The physical input material(s) 102 and output material(s), in particular machine fluid(s) and/or polyisobutene(s), 106 may be associated with one or more properties related to environmental impact. The properties related to environmental impact may be digitalized in the form of environmental attributes such as recycled or bio-based content of the input materials. The production operating system 108 may be configured to ingest such environmental attributes and to track the environmental attributes across the chemical production network 104 from input materials 102 to output material(s), in particular machine fluid(s) and/or polyisobutene(s), 106.

The chemical production network 104 may include multiple interlinked processing steps. The chemical production network 104 may be an integrated chemical production network 104 with interrelated production chains. The chemical production network 104 may include multiple different production chains that have at least one intermediate product in common. The chemical production network 104 may include multiple stages of the chemical value chain. The chemical production network 104 may include the producing, refining, processing and/or purification of gas or crude oil. The chemical production network 104 may include a stream cracker, or a syngas plant connected to multiple production chains that output products 104 from the effluent of such plants. The chemical production network 104 may include multiple production chains that output from one or more input material(s) 104 one or more output material(s), in particular machine fluid(s) and/or polyisobutene(s), 106. The chemical production network 104 may include multiple tiers of a chemical value chain. The chemical production network 104 may include a physically interconnected arrangement of production sites. The production sites may be at the same location or at different locations. In the latter case the production sites may be interconnected by means of dedicated transportation systems such as pipelines, supply chain vehicles, like trucks, supply chain ships or other cargo transportation means.

The chemical production network 104 may chemically convert input materials 102 to one or more output material(s), in particular machine fluid(s) and/or polyisobutene(s), 106. The chemical production network 104 may convert input materials 102 by way of chemical conversion to one or more output material(s), in particular machine fluid(s) and/or polyisobutene(s), 106.

The input materials 102 may be fed to the chemical production network 104 at any entry point. The input materials 102 may be fed to the chemical production network 104 at the start of the chemical production network 102. Input materials 102 may for example make up the feedstock of a steam cracker or hydrogen. The input material 102 may include non-fossil input material, such as bio-based or recycled material, and/or fossil input material for the manufacture of chemical intermediates and chemical output material(s), in particular machine fluid(s) and/or polyisobutene(s), 106.

The chemical production network 104 may include multiple production steps. The production steps included in the chemical production network 104 may be defined by the system boundary of the chemical production network 104. The system boundary may be defined by location or control over production processes. The system boundary may be defined by the site of the chemical production network 104. The system boundary may be defined by production processes controlled by one entity or multiple entities jointly. The system boundary may be defined by value chain with staggered production processes to an end product, which may be controlled by multiple entities separately. The chemical production network 104 may include a waste collection and sorting step, a recycling step such as pyrolysis, a cracking step such as steam cracking, a separation step to separate intermediates of one process step and further processing steps to convert such intermediates to output material(s), in particular machine fluid(s) and/or polyisobutene(s), 106 leaving the system boundary of the chemical production network 104.

The production operating system 108 of the chemical production network 104 may be configured to monitor and/or control the chemical production network 104 based on operating parameters of the different processes. One process step monitored and/or controlled may be the feed of input materials 102 or the release of output material(s), in particular machine fluid(s) and/or polyisobutene(s), 106. Another process step monitored and/or controlled may be the registration of environmental attributes associated with input materials 102 entering the system boundary of the chemical production network 104. Yet another process step monitored and/or controlled may be the attribution of environmental attributes to output material(s), in particular machine fluid(s) and/or polyisobutene(s), 106 produced via the chemical production network 102. Yet another process step monitored and/or controlled may be the management of environmental attributes associated with input materials 102 and output material(s), in particular machine fluid(s) and/or polyisobutene(s), 106 of the chemical production network 104.

The production operating system 108 may be configured to register inbound environmental attributes and to assign outbound environmental attributes. The production operating system 108 may be configured to access data related the inputs materials 102, the processes and/or the output material(s), in particular machine fluid(s) and/or polyisobutene(s), 106 used in the chemical production network 104. For example, the production operating system 108 may be configured to register a recycled or bio-based content of the one or more input material(s) 102 used in the chemical production network 104 as environmental attribute. The production operating system 108 may be configured to allocate the environmental attribute to at least one balancing account associated with the recycled or bio-based content of the input materials 102. The production operating system 108 may be configured to allocate at least a part of the environmental attributes from the at least one balancing account to the at least one output material(s), in particular to the machine fluid(s) and/or polyisobutene(s), 106.

The production operating system 108 may be configured to handle environmental attributes related to the input materials 102 and output material(s), in particular machine fluid(s) and/or polyisobutene(s), 106 of the chemical production network 104. For example, the production operating system 108 may be configured to determine environmental attributes associated with the use of input materials 102 impacting the environmental property of the chemical production network 104 and the output material(s), in particular machine fluid(s) and/or polyisobutene(s), 106 produced by the chemical production network 104. Further in particular, the production operating system 108 may be configured to determine environmental attributes associated with the output material(s), in particular machine fluid(s) and/or polyisobutene(s), 106. This way the production operating system 108 may be configured to store environmental attributes in balancing accounts or to withdraw environmental attributes from the balancing accounts. The environmental attributes may hence be viewed as a credit that may be deposited in an account or deducted from an account related to the input and output material(s), in particular machine fluid(s) and/or polyisobutene(s), 106 of the chemical production network 104. This way the environmental impact of the production may be tracked and/or traced.

In chemical production networks 104, multiple value chains may be linked. Additionally different input materials 102 or chemical processes impacting the environmental property of output material(s), in particular machine fluid(s) and/or polyisobutene(s), 106 produced by the chemical production network 104 may be used. Examples of input materials 102 impacting at least one environmental property of output material(s), in particular machine fluid(s) and/or polyisobutene(s), 106 produced from such input materials 102 are recycled, renewable or bio-based input materials 102. Examples of chemical processes impacting the environmental property include chemical processes using environmentally friendly technology such as carbon capture, carbon utilization or heat pumps.

Owing to the processing of chemicals in continuous or semi-continuous production and the complexity of chemical production networks 104, traceability of the input materials 102 through the network may be hampered. In such scenarios, an equivalent environmental attribute signifying the impact on the environmental property of output material(s), in particular machine fluid(s) and/or polyisobutene(s), 106 produced by the chemical production network 108 may be allocated to balancing accounts and assigned to one or more output material(s), in particular machine fluid(s) and/or polyisobutene(s), 106 of the chemical production network 104. The environmental attributes may hence be decoupled from the physical material flow inside the chemical production network 104. Decoupling may be based on the mass balance model in that the equivalent amount assigned to the one or more output material(s), in particular machine fluid(s) and/or polyisobutene(s), 106 may not exceed the equivalent amount provided by input materials 102 or processes. If an equivalent amount has been allocated to the virtual account of one environmental attribute type, it may not be allocated a second time to another virtual account of the one environmental attribute type. Environmental attribute types may be recycled, bio-based, renewable or the like. Environmental attributes may be provided in the form of digital assets or machine fluid passports attached to the physical entity of the machine fluid. Environmental attributes may be provided in the form of digital assets or polyisobutene passports attached to the physical entity of the polyisobutene.

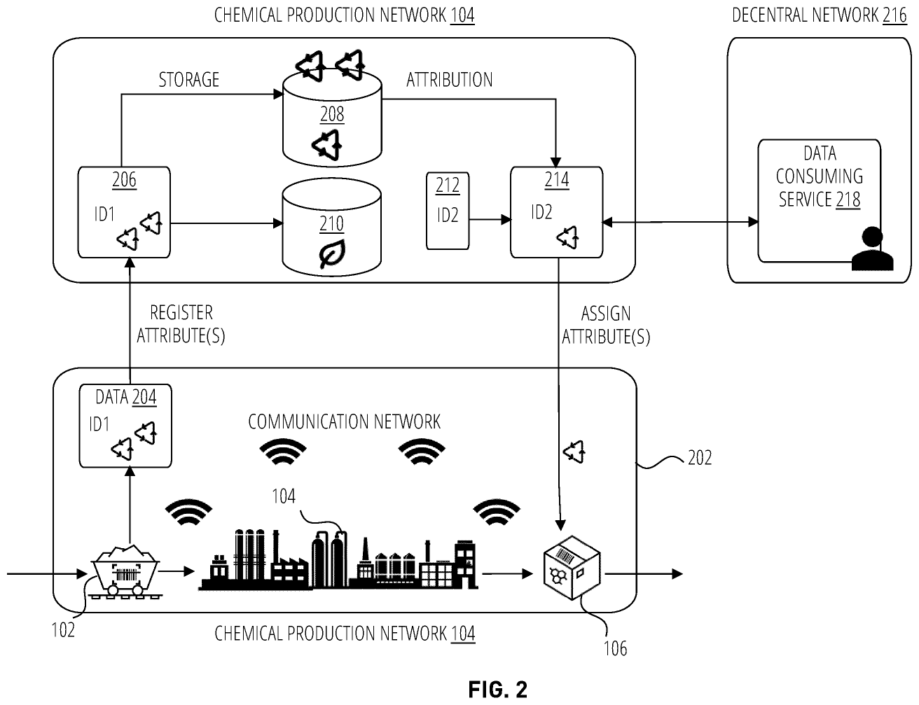

FIG. 2 illustrates schematically an example of attributing environmental attributes associated with input materials 102 to output material(s), in particular machine fluid(s) and/or polyisobutene(s), 106 of the chemical production network 104. The chemical production network 104 comprises a physical system boundary 202. The physical system boundary 202 may be defined as previously described.

As shown in FIG. 1 the chemical production network 104 and operations of the chemical production network 104 may be monitored and/or controlled by a production operating system 108. The production operating system 108 may be configured to track environmental attributes from input materials 102 fed to the chemical production network 104 to output material(s), in particular machine fluid(s) and/or polyisobutene(s), 106 produced by the chemical production network 104. For tracking, the operating system 108 may be configured to register environmental attributes associated with the input materials 102 provided to the chemical production network 104 and to attribute environmental attributes to output material(s), in particular machine fluid(s) and/or polyisobutene(s), 106 produced by the chemical production network 104.

The input materials 102 such as pyrolysis oil, bio-naphtha, bio-gas or recycled ethylene glycol, carbohydrate, lignin or fatty acid may be provided to the chemical production network 104. The input materials 102 may enter the chemical production network 104 at the entry point, such as a such as a steam cracker or a syngas plant. The input materials 102 may be used in the chemical production network 104 to produce one or more output material(s), in particular machine fluid(s) and/or polyisobutene(s), 106 from the input materials 102. Output material(s), in particular machine fluid(s) and/or polyisobutene(s), 106 may be provided at exit points of the chemical production network 104. Output material(s) may be machine fluid(s) selected from the group consisting of lubricants, lubricant additives, lubricant base oils, fuel additives, engine coolants and brake fluids. For instance, the machine fluid may be a lubricant, a lubricant additive, a lubricant base oil, a mixture of lubricant additives, a mixture of lubricant base oils, or a mixture of lubricant additives and lubricant base oils. In another instance, the machine fluid may be a fuel additive or a mixture of fuel additives. In yet another instance, the machine fluid may be an engine coolant. In yet another instance, the machine fluid may be a brake fluid. Output material(s) may be highly reactive polyisobutene(s) (HR-PIB). Output material(s) may be polyisobutene(s) having an average molecular weight (Mᵥ) (viscosity average) of 30,000 to 3,000,000 g/mol.

On entry of the input material 102, input material data 204 may be provided via a communication network to a computing interface of the production operating system 108. A data provider, such as a QR code reader, may be configured to provide material data 108 related to the one or more input material(s) 102 and respective environmental attributes 108 to a computing interface configured to allocate the environmental attributes associated with the input materials 102. The material data 108 may include the input material identifier and environmental attributes associated with the input materials 102. The input material identifier may be associated with the physical entity of the input material 102 entering the chemical production network 104. The material data may be provided on, prior or after providing of the one or more input material(s) at entry points to the chemical production network 104.

The input material identifier may be linked to the environmental attribute(s) associated with the respective input material(s) 102, the amount of input material 102 and the certificate certifying the environmental attribute(s). The amount of input material may be a measured amount of input material 102 fed to a plant or storage of the chemical production network 104 for producing one or more output material(s), in particular machine fluid(s) and/or polyisobutene(s), 106 from the input material(s) 102. The input material identifier associated with the respective input material 102, the environmental attribute(s) associated with the respective input material(s) 102 and the amount of input material(s) 102 provided to the chemical production network 104 may be provided to the production operating system 108. Such data may be provided via a communication network on entry to chemical production network 104, or the data may be transferred from a computing system to the production operating system 108.

An inbound allocator 204 may be configured to allocate the one or more environmental attribute(s) to at least one balancing account 208 associated with the respective environmental attribute. For example, one balancing account 208 may relate to environmental attributes from bio-based material and another balancing account 210 may relate to environmental attributes from recycled material. The balancing account may be associated with the respective environmental attribute type, such as bio-based or recycled. Based on such association the balancing account associated with the environmental attribute type of the respective input material 102 may be selected. The environmental attributes may be allocated to the selected balancing account. For example, the balancing account 208 for recycled material may be selected and the environmental attribute may be allocated to such account 208.

To allocate, the one or more environmental attribute(s) may be converted to balancing units and the balancing units may be allocated to the balancing account 208. The conversion may be based on a conversion factor such as mass, weight, carbon atoms, hydrogen atoms, methane equivalents or any other suitable measure for quantifying the environmental impact of the environmental attribute. The conversion factor may hence consider the difference between producing chemical products from conventional input material(s) and producing chemical products from non-conventional input material(s) or producing chemical products from a mix of conventional and non-conventional input materials. The conversion factor may relate to differences in chemical and/or physical properties of conventional and non-conventional input material(s).

By using the balancing accounts 208 it can be ensured that environmental attributes of input materials 102 are only used once for assignment to machine fluid(s) and/or polyisobutene(s) 106. This way double counting on input or output is avoided to ensure that positive environmental impact can be reliable tracked and assigned to machine fluid(s) and/or polyisobutene(s) 106.

An identifier provider 212 may be configured to provide the machine fluid identifier (ID2) associated with the machine fluid produced by the chemical production network 104 and provided at the exit point from the chemical production network 104. An identifier provider 212 may be configured to provide the polyisobutene identifier (ID2) associated with the polyisobutene produced by the chemical production network 104 and provided at the exit point from the chemical production network 104.

An outbound assigner 214 may be configured to assign at least one environmental attribute from the at least one balancing account 208 associated with the respective environmental attribute to the machine fluid identifier ID2 and/or to the polyisobutene identifier ID2. One or more environmental attribute(s) may be assigned to the at least one machine fluid identifier ID2. One or more environmental attribute(s) may be assigned to the at least one polyisobutene identifier ID2. Assignment may include de-allocation of the one or more environmental attributes from the balancing account 208 associated with the respective environmental attribute type. Assignment may include converting one or more balancing unit(s) to one or more environmental attribute(s).

Assigning at least one environmental attribute associated with input material(s) to machinal fluid(s) may include the linking of the machine fluid identifier ID2 with the environmental attribute. Assigning at least one environmental attribute associated with input material(s) to polyisobutene(s) may include the linking of the polyisobutene identifier ID2 with the environmental attribute. The machine fluid identifier ID2 may be associated with the physical entity of the machine fluid. The polyisobutene identifier ID2 may be associated with the physical entity of the polyisobutene. This way the virtual identifier of the material, such as the machine fluid and the polyisobutene, may be uniquely linked to the physical material. Such linking may include a physical or virtual link of identifiers uniquely associated with the physical material. For physical linking a tag or code may be physically connected to the material, e.g., by printing a QR code on the packaging or by embossing a code into the material. For virtual linking, different identifiers associated with the physical material may be linked. For example, an order number, a batch number, LOT number or a combination thereof may be linked.

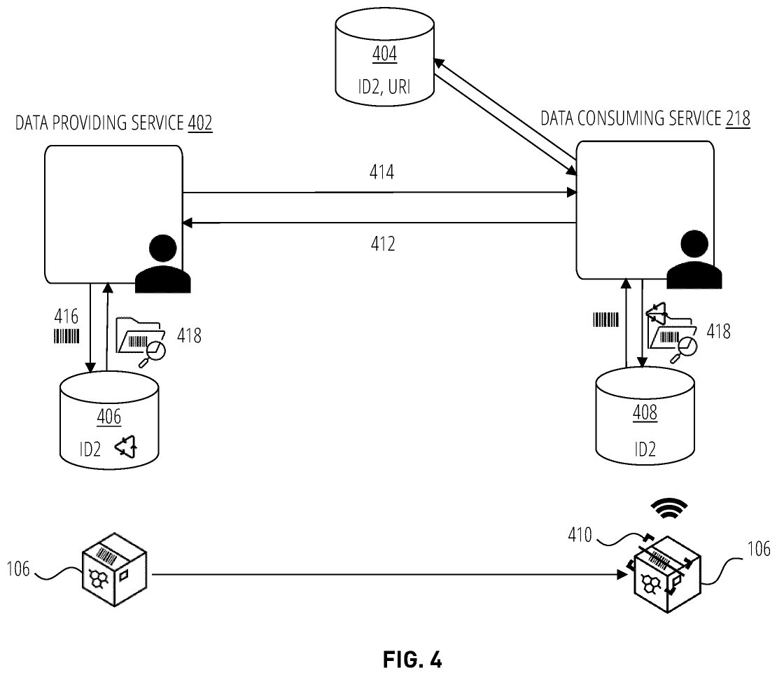

The outbound assigner 214 may be configured to provide the environmental attributes associated with the machine fluid(s) and/or the polyisobutene(s) to a data consumer, such as a data consuming service 218, associated with a consumer of the machine fluid(s) and/or the polyisobutene(s). The outbound assigner 214 may be configured to provide the environmental attributes associated with the machine fluid(s) and/or the polyisobutene(s) to a decentral network, such as decentral network 216, as will be described in the example of FIG. 4. Environmental attributes may be provided via the above ID based schema in the form of digital assets or machine fluid passports associated with the physical entity of the machine fluid. Environmental attributes may be provided via the above ID based schema in the form of digital assets or polyisobutene passports associated with the physical entity of the polyisobutene.

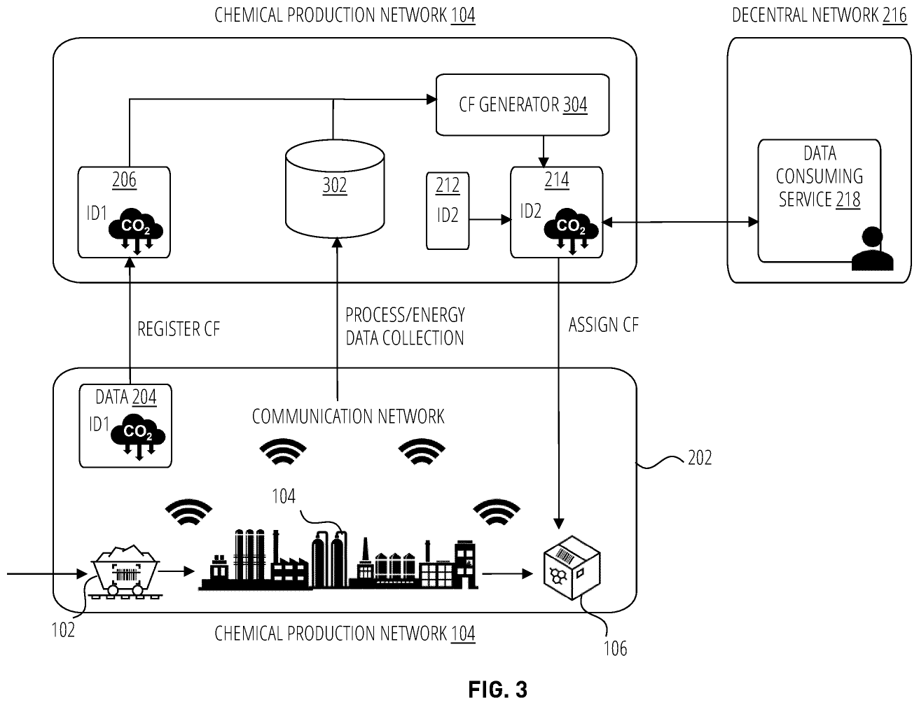

FIG. 3 illustrates schematically an example of attributing environmental attributes of input materials 102 and chemical processes to the machine fluid(s) and/or polyisobutene(s) 106 produced by the chemical production network 102. The chemical production network 104 comprises a physical system boundary 202. The physical system boundary 202 may be defined as previously described.

As described in the context of FIGS. 1 and 2 the chemical production network 104 and operations of the chemical production network 104 may be monitored and/or controlled by a production operating system 108. Input materials 102 such as pyrolysis oil, bio-naphtha or bio-gas may be provided to the chemical production network 104. The input materials 102 may be used in the chemical production network 104 to produce one or more machine fluid(s) and/or polyisobutene(s) 106 from the input materials 102.

On entry of the input material 102, input material data 204 may be provided via a communication network to a computing interface of the production operating system 108. A data provider, such as a QR code reader, may be configured to provide input material data 204 related to the one or more input material(s) 102 and respective environmental attributes to a computing interface configured to allocate the environmental attributes associated with the input materials 102. The input material data 204 may include the input material identifier and environmental attributes associated with the input materials 102. The input material identifier may be associated with the physical entity of the input material 102 entering the chemical production network 104. The input material identifier may be linked to the carbon footprint of the input material 102 as environmental attribute. The material data may be provided on, prior or after providing of the one or more input material(s) at entry points to the chemical production network 104.

The inbound allocator 206 may be configured to retrieve the one or more environmental attribute(s) and to provide such attributes to the carbon footprint (CF) generator 304. A process data provider 302 may be configured to gather process data associated with the chemical processing of the input material(s) 102 to produce the machine fluid(s) and/or polyisobutene(s) 106. The process data provider 302 may be configured to gather energy data associated with the energy consumption of the chemical processing. The process data provider 302 may be configured to provide the process data and the energy data to the CF generator 304.

The CF generator 304 may be configured to determine the carbon footprint of the machine fluid(s) and/or polyisobutene(s) produced by the chemical production network. The carbon footprint of the of the machine fluid(s) and/or polyisobutene(s) may be determined based on the process data, the energy data and the carbon footprint of the input material(s) 102 used to produce the machine fluid(s) and/or polyisobutene(s).

An identifier provider 212 may be configured to provide the machine fluid identifier associated with the machine fluid produced by the chemical production network 104 and provided at the exit point from the chemical production network 104. An identifier provider 212 may be configured to provide the polyisobutene identifier associated with the polyisobutene produced by the chemical production network 104 and provided at the exit point from the chemical production network 104.

An outbound assigner 214 may be configured to assign the determined carbon footprint to the machine fluid identifier ID2. An outbound assigner 214 may be configured to assign the determined carbon footprint to the polyisobutene identifier ID2. One or more environmental attribute(s) may be assigned to the at least one machine fluid identifier ID2, such as described in the context of FIG. 2. One or more environmental attribute(s) may be assigned to the at least one polyisobutene identifier ID2, such as described in the context of FIG. 2.

The outbound assigner 214 may be configured to provide the environmental attributes, in particular the carbon footprint, associated with the produced machine fluid(s) and/or polyisobutene(s) to a data consumer, such as a data consuming service 218, associated with a consumer of the machine fluid(s) and/or the polyisobutene(s). The outbound assigner 214 may be configured to provide the environmental attributes associated with the machine fluid(s) and/or the polyisobutene(s) to a decentral network, such as decentral network 216, as will be described in the example of FIG. 4. Environmental attributes may be provided via the above ID based schema in the form of digital assets or machine fluid passports associated with the physical entity of the machine fluid. Environmental attributes may be provided via the above ID based schema in the form of digital assets or polyisobutene passports associated with the physical entity of the polyisobutene.

FIG. 4 illustrates schematically an example of a method or apparatus for providing environmental attributes associated with machine fluid(s) and/or polyisobutene(s) to a material consumer as data consumer via a decentral network.

The machine fluid(s) and/or polyisobutene(s) 106 as produced by the chemical production network 104 may be provided in association with the digital asset as described in the context of FIGS. 2 and 3. The digital asset may include the machine fluid identifier. The digital asset may include the polyisobutene identifier. The digital asset may include one or more environmental attribute(s) such as the product carbon footprint, recycled content or bio-based content. The digital asset may relate to one or more environmental attribute(s) such as the product carbon footprint, recycled content or bio-based content. The digital asset may include a digital representation of one or more environmental attribute(s) such as the product carbon footprint, recycled content or bio-based content.

The digital asset may further include or relate to authentication and/or authorization information linked to the machine fluid identifier. The digital asset may further include or relate to authentication and/or authorization information linked to the polyisobutene identifier. The authentication and/or authorization information may be provided for authentication and/or authorization of a data providing service 402 and/or data consuming service 218. The machine fluid identifier may include or relate to a decentral identifier, that is uniquely associated with the machine fluid. The polyisobutene identifier may include or relate to a decentral identifier, that is uniquely associated with the polyisobutene. The decentral identifier may be connected to the digital representation of the environmental attributes. The digital representation may include a representation for accessing the environmental attributes or parts thereof. The decentral identifier may include a Universally Unique IDentifier (UUID) or a Digital IDentifier (DID). The decentral identifier may include any unique identifier uniquely associated with a data owner and/or the machine fluid. The decentral identifier may include any unique identifier uniquely associated with a data owner and/or the polyisobutene. The data owner may be the producer of the machine fluid(s) and/or the polyisobutene(s). Via the decentral identifier and its unique association with the data owner and/or the machine fluid(s) and/or the polyisobutene(s), access to the material configuration data may be controlled by the data owner.

The digital asset including the digital representation of one or more environmental attribute(s) such as the product carbon footprint, recycled content or bio-based content may be stored in a decentral data base 404. The one or more environmental attribute(s) such as the product carbon footprint, recycled content or bio-based content may be stored in a data base 406 associated with the data owner, such as the producer of the machine fluid(s) and/or the polyisobutene(s) 106.

The machine fluid(s) and/or the polyisobutene(s) 106 may be physically delivered to a consumer of the machine fluid(s) and/or the polyisobutene(s). The machine fluid may be connected with a QR-code or having embossed a code having encoded the machine fluid identifier. The polyisobutene may be connected with a QR-code or having embossed a code having encoded the polyisobutene identifier. The consumer of the machine fluid and/or the polyisobutene may read the code through a code reader, such as a QR-code reader, 410. The machine fluid identifier may be provided to a data base 408 associated with the consumer of the machine fluid 106. The polyisobutene identifier may be provided to a data base 408 associated with the consumer of the polyisobutene 106. In other embodiments the consumer of the machine fluid may retrieve the machine fluid identifier through the decentral data base 404. In other embodiments the consumer of the polyisobutene may retrieve the polyisobutene identifier through the decentral data base 404.

The data owner in this example may be the input material producer, the output material producer, the output material user, the end product producer. The data owner may comprise any entity generating data. The data generating node may be coupled to the data owner or the entity owning or producing physical products from or for which data is generated. The data may be generated by a third-party entity on behalf of the entity owning physical products from or for which data is generated.

The data consuming service 218 may comprise computer-executable instructions for accessing and/or processing data, such as machine fluid data and/or polyisobutene data, associated with the data owner. The data providing service 402 may comprise computer-executable instructions for providing and/or processing data, such as machine fluid data and/or polyisobutene data, associated with the data owner for accessing and/or processing by the data consuming service 218.

Based on the received machine fluid identifier a request to access the environmental attributes associated with the machine fluid identifier may be triggered by the data consuming service 218 as signified by arrow 412. The machine fluid identifier may be provided to the data providing service 402 associated with or of the producer of the machine fluid 102. Based on the received polyisobutene identifier a request to access the environmental attributes associated with the polyisobutene identifier may be triggered by the data consuming service 218 as signified by arrow 412. The polyisobutene identifier may be provided to the data providing service 402 associated with or of the producer of the polyisobutene 102. In addition, authentication and/or authorization information may be provided.

The request may be authenticated and/or authorized to access the environmental attributes associated with the machine fluid identifier. Based on successful authorization and/or authentication, access to the environmental attributes associated with the machine fluid identifier may be granted. The request may be authenticated and/or authorized to access the environmental attributes associated with the polyisobutene identifier. Based on successful authorization and/or authentication, access to the environmental attributes associated with the polyisobutene identifier may be granted.

For access, the machine fluid identifier and/or the polyisobutene identifier may be provided to the data providing service 402 as signified by arrow 412. The data providing service 402 may use the received machine fluid identifier to retrieve the environmental attributes associated with the received machine fluid identifier as signified by arrows 416 and 418. The data providing service 402 may use the received polyisobutene identifier to retrieve the environmental attributes associated with the received polyisobutene identifier as signified by arrows 416 and 418. The retrieved environmental attributes associated with received machine fluid identifier and/or the received polyisobutene identifier may be provided to the data consuming service 218 as signified by arrow 414. The environmental attributes associated with the machine fluid 106 may be stored in the data base 408 associated with the consumer of the machine fluid 106 as signified by arrow 418. The environmental attributes associated with the polyisobutene 106 may be stored in the data base 408 associated with the consumer of the polyisobutene 106 as signified by arrow 418.

Through the output identifier or decentral identifier, the environmental attributes can be uniquely associated with the machine fluid. Through the output identifier or decentral identifier, the environmental attributes can be uniquely associated with the polyisobutene. Through the decentral network the environmental attributes may be transferred between the producer of the machine fluid and/or the polyisobutene and the consumer of the machine fluid and/or the polyisobutene. This way the environmental attributes can be shared with unique association to the machine fluid and/or the polyisobutene and without central intermediary directly between the value chain players. This allows for transparency of environmental attributes across the value chain and positive environmental impacts from machine fluid(s) and/or the polyisobutene(s) produced by the chemical production network 104 can be tracked through the value chain.

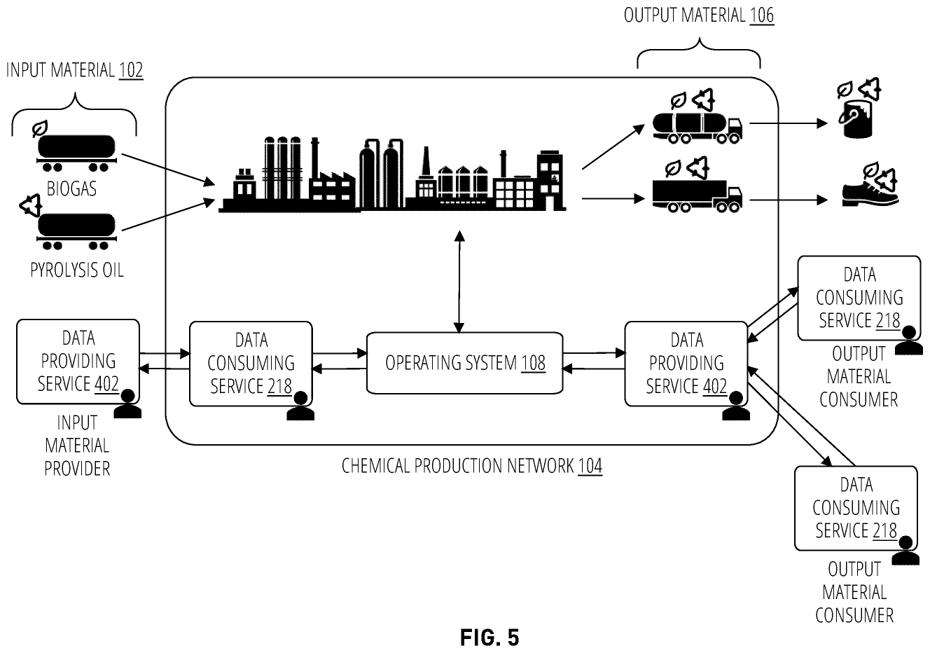

FIG. 5 illustrates schematically an example of a method or apparatus for providing environmental attributes associated with machine fluid(s) and/or polyisobutene(s) across value chains via the decentral network.

In the example of FIG. 5 a fully connected value chain including the chemical production network 104 is illustrated. In the example, the input material provider, the machine fluid producer, the machine fluid consumer and the end product producer may be connected to the decentral network as described in the context of FIG. 4. In the example, the input material provider, the polyisobutene producer, the polyisobutene consumer and the end product producer may be connected to the decentral network as described in the context of FIG. 4. Environmental attributes may be provided via the ID based schema described in the context of FIGS. 2 to 4 in the form of machine fluid passports or digital assets associated with the physical entity of the input material, the machine fluid, any intermediate product or the end product. Environmental attributes may be provided via the ID based schema described in the context of FIGS. 2 to 4 in the form of polyisobutene passports or digital assets associated with the physical entity of the input material, the polyisobutene, any intermediate product or the end product.

The input material provider may provide the input material 102. The input material 102 may include bio-gas, biomass, bio-based input material or recycled material. The environmental attributes of said input material 102 may be provided through the data providing service connected to the decentral network as described in the context of FIG. 4. The machine fluid producer may produce the machine fluid and/or the polyisobutene from the input material(s) 506 provided to the chemical production network 104. The machine fluid and/or polyisobutene producer may access the environmental attributes associated with the input material 102 through a data consuming service 218 connected to the decentral network as described in the context of FIG. 4. Said environmental attributes may be retrieved from a data providing service 402 associated with the respective input material provider. The machine fluid and/or polyisobutene producer may manage the environmental attributes via the production operating system 108 as described in the context of FIGS. 1 to 3. The machine fluid producer may assign the environmental attributes associated with the input materials 102 or environmental attributes associated with the chemical production network 104 such as the carbon footprint, to the produced machine fluid(s) 106 as described in the context of FIGS. 1 to 3. The polyisobutene producer may assign the environmental attributes associated with the input materials 102 or environmental attributes associated with the chemical production network 104 such as the carbon footprint, to the produced polyisobutene(s) 106 as described in the context of FIGS. 1 to 3. The machine fluid and/or polyisobutene producer may provide the environmental attributes associated with the produced machine fluid(s) 106 and/or the environmental attributes associated with the produced polyisobutene(s) 106 through the data providing service 402 connected to the decentral network as described in the context of FIG. 4. The machine fluid consumer or the end product producer may access the environmental attributes associated with the produced machine fluid(s) 106 through the data consuming service 218 connected to the decentral network as described in the context of FIG. 4. The polyisobutene consumer or the end product producer may access the environmental attributes associated with the produced polyisobutene(s) 106 through the data consuming service 218 connected to the decentral network as described in the context of FIG. 4.

The respective data owners in this example may be the input material producer, the output material producer, the output material consumer, the end product producer. The data owner may comprise any entity generating data. The data generating node may be coupled to the data owner or the entity owning or producing physical products from or for which data is generated. The data may be generated by a third-party entity on behalf of the entity owning physical products from or for which data is generated.

The data consuming service 218 may comprise computer-executable instructions for accessing and/or processing data, such as machine fluid and/or polyisobutene data, associated with the data owner. The data providing service 402 may comprise computer-executable instructions for providing and/or processing data, such as machine fluid and/or polyisobutene data, associated with the data owner for accessing and/or processing by the data consuming service.

In the example of FIG. 5 the decentral identifier may relate to the end product. Such decentral identifier may be provided to the value chain participants. Via the end product specific decentral identifier data associated with the end product produced from the machine fluid and/or produced from the polyisobutene may be gathered across the production chain and assigned to the end product specific decentral identifier. For example, the one or more environmental attribute(s) associated with the end product may be derived from the environmental attribute(s) associated with the machine fluid and/or derived from the environmental attribute(s) associated with the polyisobutene, the input material 102 or any other product entity present in the value chain of the end product.

This way the environmental attributes of input materials 102, produced machine fluid(s) and/or produced polyisobutene(s) 106 and any products produced from said machine fluid(s) and/or produced polyisobutene(s) 106 may be tracked through the value chain up to the end product. By tracking the environmental attributes of materials in such way the information can be made transparent across the value chain while the information flow can be controlled by the participants in the supply chain. In addition, the environmental attributes can be handled according to the individual participants needs by production operating systems as described in the context of FIGS. 1 to 3. Overall, such tracking enables tracking of positive environmental impact by individual supply chain participants, which makes positive environmental impacts transparent and attributable to individual supply chain participants.

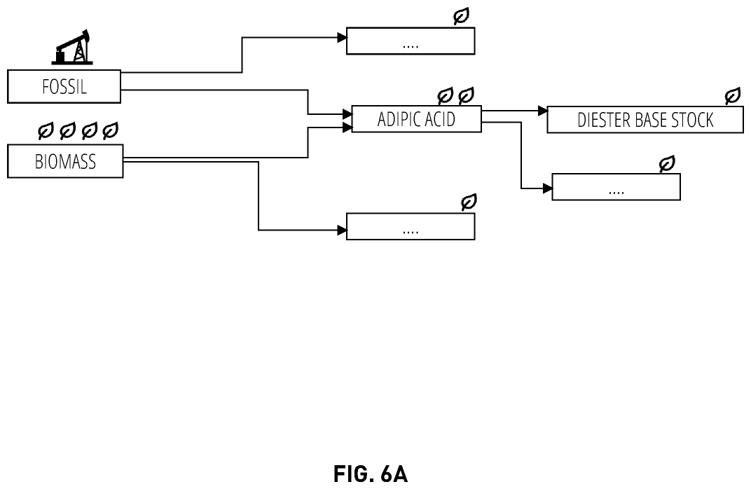

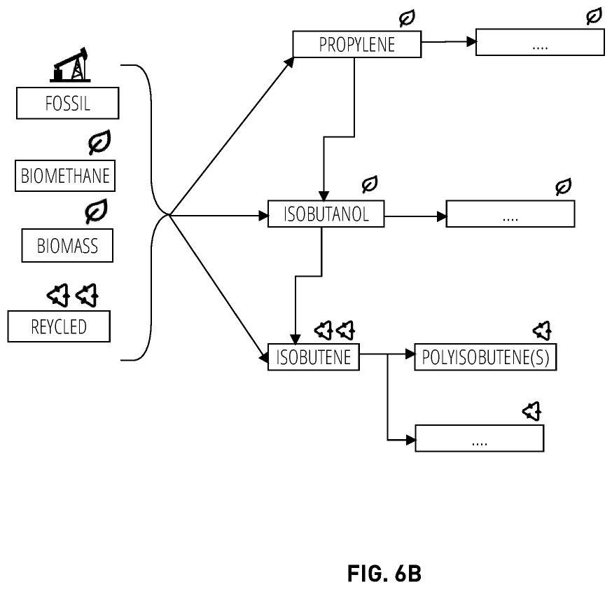

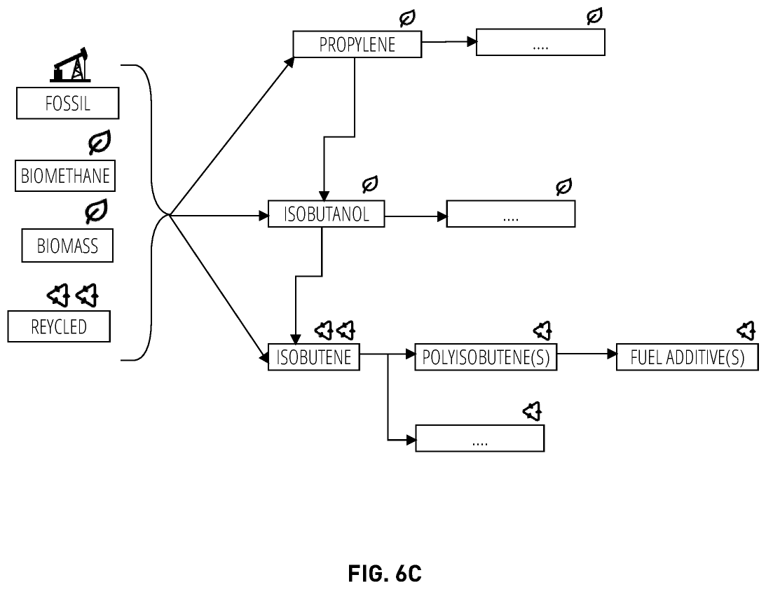

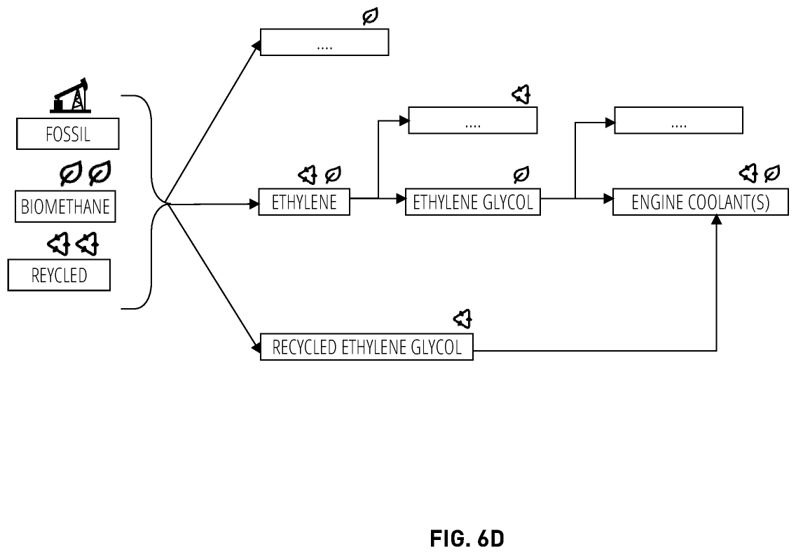

FIG. 6A illustrates schematically an example of a chemical production network for producing lubricant base oil(s), such as diester base stock, associated with a machine fluid (e.g. lubricant base oil) passport or digital asset. The example of FIG. 6A is an illustrative example and should not be considered limiting.

In the example of FIG. 6A, fossil feedstock and bio feedstock may be provided to the chemical production network for producing as an example lubricant base oils in the form of diester base stock (see for example FIGS. 1 to 5). The fossil feedstock may include natural gas. The bio feedstock may include biomass, such as carbohydrate(s), lignin or fatty acid. The lubricant base oils may be used to produce lubricants, for example by mixing the lubricant base oil with one or more lubricant additives. Natural gas and biomass may be the input materials in this example as mixed feedstock. From natural gas and biomass, adipic acid and other intermediate materials may be produced. From adipic acid, diester base stock material(s) and other products, such as intermediate materials or output materials, may be produced.

In the example of FIG. 6A the bio-based content of biomass as non-fossil input material may be tracked. As described in the context of FIG. 2, the bio-based content at the start of the diester base stock production chain may be registered via the production operating system 108 (not shown) of the chemical production network 104 (not shown). Here the bio-based content may be allocated to the balancing account 208 for bio-based materials. The environmental attribute of bio-based content is hence detached from the mass flows of the chemical processing in the chemical production network 104 as illustrated in FIG. 6A.

For attribution of the biomass content used to produce the diester base stock, the biomass may be determined. The biomass content attributable to diester base stock production may be based on mass conservation attributable to the produced diester base stock. For example, only a part of the biomass content may be attributable to the diester base stock while the other part may be attributable to other output products resulting from the biomass as input material. The environmental attribute bio-based content may be attributed to such extend to the produced diester base stock.

The registration of the biomass content of the input materials as well as the attribution of said registered environmental attributes to the produced diester base stock may be performed as described in relation to FIG. 2. In the system shown in FIG. 2, the environmental attribute associated with the production of the diester base stock may be attributed to the diester base stock by associating the decentral ID to the diester base stock and assigning the environmental attribute to the decentral ID. By linking the ID and the environmental attribute, the environmental attribute may be uniquely linked to the produced diester base stock. The produced diester base stock may be delivered to a diester base stock consumer. The packaging, such as containers or tanks, may include a code, such as an QR-code or an embossed code. The decentral ID may be included or encoded to said code. This way the diester base stock consumer may access the environmental attributes associated to the produced diester base stock via the ID based protocol described in the context of FIGS. 4 and 5.

Similarly to this example, the registration of carbon footprint data associated with input materials and production processes as well as the attribution of said carbon footprint to the produced diester base stock may be based on the method illustrated in FIG. 3 for carbon footprints.

FIG. 6B illustrates schematically a further example of a chemical production network 104 for producing polyisobutene(s) associated with a polyisobutene passport or digital asset. The example of FIG. 6B is an illustrative example and should not be considered limiting.

In the example of FIG. 6B, fossil feedstock, bio feedstock and recycled feedstock may be provided to the chemical production network 104 (see for example FIGS. 1 to 5) for producing as an example polyisobutene(s). The polyisobutene(s) may be a highly reactive polyisobutene(s). The polyisobutene(s) may be polyisobutene(s) having an average molecular weight (Mᵥ) (viscosity average) of 30,000 to 3,000,000 g/mol. The fossil feedstock may include natural gas. The bio feedstock may include biomass, such as carbohydrate(s), lignin or fatty acid. The bio feedstock may include biomethane. The bio feedstock may include a mixture of biomass and biomethane. The recycled feedstock may include pyrolysis oil. Natural gas, biomass and pyrolysis oil may be the input materials in this example as mixed feedstock. From the mixed feedstock, propylene and other intermediate materials may be produced. The propylene may be used to produce isobutanol. From the mixed feedstock, isobutanol and other intermediate materials may be produced. The isobutanol may be used to produce isobutene. Isobutene may also be produced, along with other products, such as intermediate materials or output materials, from the mixed feedstock. The isobutene may be used as intermediate product to produce the polyisobutene(s), such as the polyisobutene(s) previously mentioned.

In the example of FIG. 6B, the biomethane content of biomethane, the bio-content of the biomass and/or the pyrolysis oil content of the pyrolysis oil as non-fossil input material may be tracked. As described in the context of FIG. 2, the biomethane content and/or the pyrolysis oil content and/or the biomass content at the start of the polyisobutene production chain may be registered via the operating system 108 (not shown) of the chemical production network 104. Here the pyrolysis oil content may be allocated to the balancing account 210 associated with metadata, such as metadata indicating that this balancing account is assigned to recycled materials. The biomethane content and the biomass content may be allocated to the balancing account 208 associated with metadata, such as metadata indicating that this balancing account is assigned to bio-based materials. The environmental attribute of biomethane content and/or biomass content and/or pyrolysis oil content is hence detached from the mass flows of the chemical processing in the chemical production network 104 as illustrated in FIG. 6B.

For attribution of the biomethane content and/or the biomass content and/or the pyrolysis oil content used to produce the polyisobutene(s), the biomethane content and/or the biomass content and/or the pyrolysis oil content may be determined. The biomethane content and/or the biomass content and/or the pyrolysis oil content attributable to the polyisobutene production may be based on mass conservation attributable to the produced polyisobutene(s). For example, only half of the pyrolysis oil content may be attributable to polyisobutene(s) while the remaining biomethane, biomass and pyrolysis oil content may be attributable to other output products resulting from the mixed feedstock as input material. The environmental attribute biomethane content and/or biomass content and/or the pyrolysis oil content may be attributed to such extend to the produced polyisobutene(s).

The registration of the biomethane content and/or the biomass content and/or the pyrolysis oil content of input materials (e.g. fossil feedstock, biomethane, recycled feedstock) as well as the attribution of said registered environmental attributes to the produced polyisobutene(s) may be performed as described in relation to FIG. 2. In the system shown in FIG. 2, the environmental attribute associated with the production of the polyisobutene(s) may be attributed to the produced polyisobutene by associating a decentral ID to the polyisobutene and assigning the environmental attribute to the decentral ID. By linking the ID and the environmental attribute, the environmental attribute may be uniquely linked to the produced polyisobutene. The polyisobutene may be delivered to the consumer of the polyisobutene, such as a production producing further products using the received polyisobutene, for example lubricants. The packaging, such containers and/or tanks, containing the polyisobutene may include a code, such as an QR-code or an embossed code. The decentral ID may be included or encoded to said code. This way the consumer of the polyisobutene may access the environmental attributes associated to the polyisobutene via the ID based protocol described in the context of FIGS. 4 and 5.

Similarly to this example the registration of carbon footprint data associated with input materials and production processes as well as the attribution of said carbon footprint to the produced polyisobutene(s) may be based on the method illustrated in FIG. 3 for carbon footprints.

FIG. 6C illustrates schematically a further example of a chemical production network for producing fuel additives, such as polyisobutene amine(s) and/or polyisobutene succinic acid anhydride (PIBSA), associated with a machine fluid (e.g. fuel additive) passport or digital asset. The example of FIG. 6C is an illustrative example and should not be considered limiting.

In the example of FIG. 6C, fossil feedstock, bio feedstock and recycled feedstock may be provided to the chemical production network (see for example FIGS. 1 to 5) for producing as an example fuel additives. The fuel additive may be polyisobutene amine(s). The fuel additive may be polyisobutene succinic acid anhydride (PIBSA). The fossil feedstock may include natural gas. The bio feedstock may include biomass, such as carbohydrate(s), lignin or fatty acid. The bio feedstock may include biomethane. The bio feedstock may include a mixture of biomass and biomethane. The recycled feedstock may include pyrolysis oil. Natural gas, biomass and pyrolysis oil may be the input materials in this example as mixed feedstock. From the mixed feedstock, propylene and other intermediate materials may be produced. The propylene may be used to produce isobutanol. From the mixed feedstock, isobutanol and other intermediate materials may be produced. The isobutanol may be used to produce isobutene. Isobutene may also be produced, along with other products, such as intermediate materials or output materials, from the mixed feedstock. The isobutene may be used as intermediate product to produce polyisobutene(s). The polyisobutene(s) may be used to produce the fuel additives, such as the amine and anhydrides previously mentioned.

In the example of FIG. 6C, the biomethane content of biomethane, the bio-content of the biomass and/or the pyrolysis oil content of the pyrolysis oil as non-fossil input material may be tracked as described in relation to FIG. 6B above.

The biomethane content and/or the biomass content and/or the pyrolysis oil content attributable to the fuel additive production may be determined as described in relation to FIG. 6B.

The registration of the biomethane content and/or the biomass content and/or the pyrolysis oil content of input materials (e.g. fossil feedstock, biomethane, recycled feedstock) as well as the attribution of said registered environmental attributes to the produced polyisobutene(s) may be performed as described in relation to FIGS. 2 and 6B by associating a decentral ID to the respective fuel additive and assigning the environmental attribute to the decentral ID.

Similarly to this example the registration of carbon footprint data associated with input materials and production processes as well as the attribution of said carbon footprint to the produced fuel additive(s) may be based on the method illustrated in FIG. 3 for carbon footprints.

FIG. 6D illustrates schematically a further example of a chemical production network 104 for producing engine coolants, such as engine coolants based on ethylene glycol, associated with a machine fluid (e.g. engine coolant) passport or digital asset. The example of FIG. 6D is an illustrative example and should not be considered limiting.

In the example of FIG. 6D, fossil feedstock, biomethane and recycled feedstock may be provided to the chemical production network (see for example FIGS. 1 to 5) for producing as an example engine coolant(s). The engine coolant(s) may include ethylene glycol. The engine coolant(s) may further include water and/or additives. The fossil feedstock may include natural gas. The bio feedstock may include biomethane. The recycled feedstock may include pyrolysis oil. The recycled feedstock may include recycled ethylene glycol. The recycled ethylene glycol may be obtained from waste engine coolant. The waste engine coolant may be recycled within the chemical production network 104 to obtain recycled ethylene glycol. The recycled feedstock may include pyrolysis oil and recycled ethylene glycol. Natural gas, biomethane, recycled ethylene glycol and/or pyrolysis oil may be the input materials in this example as mixed feedstock. From the mixed feedstock, ethylene and other intermediate materials or output materials may be produced. The ethylene may be used as intermediate material to produce ethylene glycol. The ethylene glycol may be used as intermediate material to produce the engine coolant(s) and other intermediate materials or output materials. The recycled ethylene glycol may be used to produce the engine coolant(s). The recycled ethylene glycol may be mixed with ethylene glycol produced from ethylene. The recycled ethylene glycol may not be mixed with ethylene glycol produced from ethylene.

In the example of FIG. 6D, the biomethane content of biomethane and/or the pyrolysis oil content of the pyrolysis oil and/or the content of recycled ethylene glycol as non-fossil input material may be tracked as described in relation to FIGS. 6B and 6C above.

The biomethane content and/or the content of recycled ethylene glycol and/or the pyrolysis oil content attributable to the engine coolant production may be determined as described in relation to FIGS. 6B and 6C.

The registration of the biomethane content and/or the content of recycled ethylene glycol and/or the pyrolysis oil content of input materials (e.g. fossil feedstock, biomethane, recycled feedstock, waste feedstock) as well as the attribution of said registered environmental attributes to the produced engine coolants(s) may be performed as described in relation to FIGS. 2, 6B and 6C by associating a decentral ID to the respective engine coolant and assigning the environmental attribute to the decentral ID.

Similarly to this example the registration of carbon footprint data associated with input materials and production processes as well as the attribution of said carbon footprint to the produced engine coolant(s) may be based on the method illustrated in FIG. 3 for carbon footprints.

The present disclosure has been described in conjunction with preferred embodiments and examples as well. However, other variations can be understood and effected by those persons skilled in the art and practicing the claimed invention, from the studies of the drawings, this disclosure and the claims.

Any steps presented herein can be performed in any order. The methods disclosed herein are not limited to a specific order of these steps. It is also not required that the different steps are performed at a certain place or in a certain computing node of a distributed system, i.e. each of the steps may be performed at different computing nodes using different equipment/data processing.

As used herein “determining” also includes “initiating or causing to determine”, “generating” also includes “initiating and/or causing to generate” and “providing” also includes “initiating or causing to determine, generate, select, send and/or receive”. “Initiating or causing to perform an action” includes any processing signal that triggers a computing node or device to perform the respective action.

In the claims as well as in the description the word “comprising” does not exclude other elements or steps and the indefinite article “a” or “an” does not exclude a plurality. A single element or other unit may fulfill the functions of several entities or items recited in the claims. The mere fact that certain measures are recited in the mutual different dependent claims does not indicate that a combination of these measures cannot be used in an advantageous implementation.

1. A system for producing a machine fluid associated with a machine fluid passport or a digital asset and/or a polyisobutene associated with a polyisobutene passport or a digital asset, the system comprising:
(a) a chemical production network configured to produce a machine fluid and/or a polyisobutene from one or more input material(s) through one or more chemical process(es) of the chemical production network, wherein the one or more input material(s) and/or the one or more chemical process(es) are associated with one or more environmental attribute(s):
(b) a production operating apparatus configured to generate a machine fluid passport or a machine fluid digital asset and/or a polyisobutene passport or a polyisobutene digital asset by
(i) providing a decentral identifier, wherein the decentral identifier is associated with the machine fluid and the one or more environmental attribute(s) of the one or more input material(s) and/or the one or more chemical process(es); and/or the decentral identifier is associated with the polyisobutene and the one or more environmental attribute(s) of the one or more input material(s) and/or the one or more chemical process(es); and
(ii) linking the decentral identifier and the one or more environmental attribute(s); and
(c) the chemical production network or a chemical production system configured to provide the machine fluid in association with the machine fluid passport or the machine fluid digital asset and/or to provide the polyisobutene in association with the polyisobutene passport or the polyisobutene digital asset.

2. The system of claim 1, wherein the machine fluid associated with the machine fluid passport or the machine fluid digital asset is at least one selected from the group consisting of lubricants, lubricant additives, lubricant base oils, fuel additives, engine coolants and brake fluids; and/or
wherein the polyisobutene associated with the polyisobutene passport or the polyisobutene digital asset is a polyisobutene having a number average molecular weight Mₙ from 500 to 1,000,000 g/mol as measured by gel permeation chromatography.

3. The system of claim 1, wherein the machine fluid passport or the machine fluid digital asset associated with the machine fluid comprises mass balanced environmental attributes related to the one or more input material(s), and/or
wherein the polyisobutene passport or the polyisobutene digital asset associated with the polyisobutene includes mass balanced environmental attributes related to the one or more input material(s).

4. The system of claim 1, wherein the one or more environmental attribute(s) associated with the machine fluid and/or the polyisobutene are provided from at least one balancing account configured to store the one or more environmental attribute(s) associated with the one or more input material(s).

5. The system of claim 1, wherein the one or more environmental attribute(s) are associated with at least one property related to an environmental impact of the one or more input material(s) and/or the one or more chemical process(es).

6. The system of claim 1, wherein the production operating apparatus is configured to gather the one or more environmental attributes associated with the machine fluid and/or the polyisobutene before, during and/or after production of the machine fluid and/or the polyisobutene by the chemical production network.

7. The system of claim 1, wherein the one or more environmental attribute(s) associated with the machine fluid and/or the polyisobutene produced through the one or more chemical processes from one or more input material(s) provided to the chemical production network include the one or more environmental attribute(s) associated with the one or more input material(s), the one or more chemical process(es) and/or the chemical production network(s).

8. The system of claim 1, wherein the one or more environmental attribute(s) associated with the one or more input material(s) are provided before, during and/or after production of the machine fluid and/or the polyisobutene by the chemical production network, wherein the one or more environmental attribute(s) associated with the one or more input material(s) are allocated to at least one balancing account before, during and/or after production of the machine fluid and/or the polyisobutene by the chemical production network.

9. The system of claim 1, wherein the one or more environmental attribute(s) associated with the machine fluid and/or the polyisobutene relate to environmental properties generated from process data associated with chemical processing of the one or more input material(s) and/or energy data associated with energy consumption of the chemical processing, wherein the one or more environmental attribute(s) associated with the machine fluid and/or the polyisobutene are generated before, during and/or after production of the machine fluid and/or the polyisobutene by the chemical production network.

10. A method for producing a machine fluid associated with a machine fluid passport or a machine fluid digital asset and/or for producing a polyisobutene associated with a polyisobutene passport or a polyisobutene digital asset, wherein the method comprises:
(a) producing a machine fluid and/or a polyisobutene from one or more input material(s) through one or more chemical process(es) of a chemical production network. wherein the one or more input material(s) and/or the one or more chemical process(es) are associated with one or more environmental attribute(s);
(b) generating a machine fluid passport or a machine fluid digital asset and/or a polyisobutene passport or a polyisobutene digital asset by
(i) providing a decentral identifier, wherein the decentral identifier is associated with the machine fluid and the one or more environmental attribute(s) associated with the one or more input material(s) and/or the one or more chemical process(es); and/or the decentral identifier is associated with the polyisobutene and the one or more environmental attribute(s) of the one or more input material(s) and/or the one or more chemical process(es); and
(ii) linking the decentral identifier and the one or more environmental attribute(s);
(c) providing the machine fluid in association with the machine fluid passport or the machine fluid digital asset and/or providing the polyisobutene in association with the polyisobutene passport or the polyisobutene digital asset.

11. A machine fluid associated with a machine fluid passport or a machine fluid digital asset and/or a polyisobutene associated with a polyisobutene passport or a polyisobutene digital asset, wherein the passports or the digital assets comprise a decentral identifier, wherein the decentral identifier is associated with the machine fluid and linked to one or more environmental attribute(s) of one or more input material(s) and/or one or more chemical process(es) used to produce the machine fluid, or the decentral identifier is associated with the polyisobutene and linked to one or more environmental attribute(s) of one or more input material(s) and/or one or more chemical process(es) used to produce the polyisobutene.

12. The machine fluid and/or polyisobutene of claim 11, wherein the machine fluid is selected from the group consisting of lubricants, lubricant additives, lubricant base oils, fuel additives, engine coolants and brake fluids; and/or wherein the polyisobutene is a polyisobutene having a number average molecular weight Mₙ from 500 to 1,000,000 g/mol as measured by gel permeation chromatography.

13. A method for generating a machine fluid passport or a machine fluid digital asset associated with a machine fluid and/or a polyisobutene passport or a polyisobutene digital asset associated with a polyisobutene, wherein the machine fluid and/or the polyisobutene is produced from one or more input material(s) through one or more chemical process(es) of a chemical production network, wherein the one or more input material(s) and/or the one or more chemical process(es) are associated with environmental attribute(s), the method comprising:
(a) providing a decentral identifier, wherein the decentral identified is associated with the machine fluid and one or more environmental attribute(s) of the one or more input material(s) and/or the one or more chemical process(es) used to produce the machine fluid and/or the decentral identifier is associated with the polyisobutene and one or more environmental attribute(s) of the one or more input material(s) and/or the one or more chemical process(es) used to produce the polyisobutene;
(b) linking the decentral identifier and the environmental attribute(s); and
(c) providing the machine fluid passport or the machine fluid digital asset in association with the machine fluid to a decentral network, wherein the environmental attribute(s) associated with the machine fluid is made accessible to a consumer of the machine fluid through the machine fluid passport or the machine fluid digital asset and/or providing the polyisobutene passport or the polyisobutene digital asset in association with the polyisobutene to the decentral network, wherein the environmental attribute(s) associated with the polyisobutene is made accessible to a consumer of the polyisobutene through the polyisobutene passport or the polyisobutene digital asset.

14. The digital asset as generated according to the method of claim 13.

15. A method, comprising:
producing a product comprising the machine fluid associated with the machine fluid digital asset and/or the polyisobutene associated with the polyisobutene digital asset generated according to the method of claim 13 and
deriving a digital asset associated with the product from the machine fluid digital asset and/or polyisobutene digital asset.

16. The system of claim 2, wherein the machine fluid associated with the machine fluid passport or the machine fluid digital asset is at least one selected from the group consisting of a lubricant, a lubricant additive, a lubricant base oil, a mixture of lubricant additives, a mixture of lubricant base oils, and a mixture of lubricant additives and lubricant base oils.

17. The machine fluid and/or polyisobutene of claim 12, wherein the machine fluid is selected from the group consisting of a lubricant, a lubricant additive, a lubricant base oil, a mixture of lubricant additives, a mixture of lubricant base oils, and a mixture of lubricant additives and lubricant base oils.
